(19) United States
(12) Patent Application Publication
(10) Pub. No.: US 2023/0226089 A1
(43) Pub. Date: Jul. 20, 2023

(54) METHODS AND COMPOSITIONS FOR TREATING DISEASES ASSOCIATED WITH SENESCENCE

(71) Applicant: Atropos Therapeutics, Inc., San Carlos, CA (US)

(72) Inventors: Andrew KOFF, Westbury, NY (US); Thomas B. NGUYEN, Missouri City, TX (US)

(21) Appl. No.: 18/186,412

(22) Filed: Mar. 20, 2023

Related U.S. Application Data
(63) Continuation of application No. 16/734,170, filed on Jan. 3, 2020.
(60) Provisional application No. 62/788,496, filed on Jan. 4, 2019.

(51) Int. Cl. A61K 31/704; A61K 35/28; A61K 36/258; A61K 36/53; A61K 36/488; A61P 35/00; A61K 31/4709; A61K 36/79; A61K 36/70; A61K 36/804; A61K 31/519; A61K 31/65; A61K 36/815

(52) U.S. Cl. CPC A61K 31/704; A61K 35/28; A61K 36/258; A61K 36/53; A61K 36/488; A61P 35/00; A61K 31/4709; A61K 36/79; A61K 36/70; A61K 36/804; A61K 31/519; A61K 31/65; A61K 36/815

(57) ABSTRACT

The present disclosure relates to a method of treating or delaying onset of a disease associated with the accumulation of senescent cells in a subject comprising administering to the subject a therapeutically effective amount of a senosuppressor that modulates a number of ATRX foci per cell.

### CROSS-REFERENCE

This application is a continuation of U.S. patent application Ser. No. 16/734,170, filed Jan. 3, 2020, which claims the benefit of U.S. Provisional Application No. 62/788,496, filed on Jan. 4, 2019, which application is incorporated herein by reference in its entirety.

### STATEMENT AS TO FEDERALLY SPONSORED RESEARCH

This invention was made with government support under 1R43AG061998-01 awarded by SBIR from National Institute of Aging (NIA). The government has certain rights in the invention.

### BACKGROUND OF THE INVENTION

As humans age a variety of morbidities and diseases accumulate. While modern medicine has extended average lifespan, individuals spend more of their lives with multiple chronic morbidities. The social and economic consequences of an increasingly aged and infirm population are severe. There is thus a need for therapies that treat the causes and symptoms of aging.

### SUMMARY OF THE INVENTION

Disclosed herein are methods for treating or delaying onset of a disease associated with the accumulation of senescent cells in a subject comprising administering to the subject a therapeutically effective amount of a senosuppressor that modulates a number of ATRX foci per cell. In some embodiments, the number of ATRX foci per cell is a measurement of a total number of ATRX foci per cell in a sample derived from the subject after the subject was administered a quiescence-inducing compound relative to a total number of ATRX foci per cell in a reference sample collected from the subject before the administering of the senosuppressor and after the administering of the quiescence-inducing compound. In some embodiments, the senosuppressor reduces the total number of ATRX foci per cell in at least 30% of cells in the sample derived from the subject relative to the reference sample. In some embodiments, the total number of ATRX foci per cell in a sample derived from the subject is reduced at least 3-fold relative to the reference sample. In some embodiments, the number of ATRX foci per cell is a measurement of a mean number of ATRX foci per cell in a sample derived from the subject after the subject was administered a quiescence-inducing compound relative to a mean number of ATRX foci per cell in a reference sample collected from the subject before the administering of the senosuppressor and after the administering of the quiescence-inducing compound. In some embodiments, 30% of cells in the sample derived from the subject have a total number of ATRX foci per cell that is below the mean number of ATRX foci per cell in the reference sample. In some embodiments, the mean number of ATRX foci per cell in the sample derived from the subject is reduced at least 3-fold relative to the reference sample. In some embodiments, the senosuppressor comprises an extract from a plant species selected from the group consisting of Albizia julibrissin Durazz, Arisaema, Arnebia, Lithospermum, Atractylodes macrocephala, Cnidium monnieri, Dimocarpus longan Lour, Forsythia suspensa Juncus effuses, Lilium, Lophatherum gracile Brongn, Nelumbo nucifera Gaertn, Pinellia ternate, Polygonum multiflorum, and Tribulus terrestris. In some embodiments, the senosuppressor comprises an extract from a species selected from the group consisting of Radix Ginseng, Radix Astragali, Fructus Viticis, Radix Puerariae, Cortex Phellodendri, Rhizoma Cimicifugae, Radix Glycyrrhizae, Rhizoma Acori Graminei, Radix Polygalae Tenuifoliae, Sclerotium Poriae Cocos, Scierotium Pararadicis Poriae Cocos, Semen Cuscutae Chinensis, Cortex Radicis Lycii Chinensis, Fructus Schisandrae Chinensis, Rhizoma Ligustici Chuanxiong, Radix Rehmanniea, Fructus Corni Officinalis, Semen Zizyphi Spinosae, Plastrum Testudinis, Os Draconis, Radix Rehmanniae Glutinosae, Tuber Ophiopogonis Japonici, Tuber Asparagi Cochinensis, Radix Angelicae Sinensis, Fructus Psoraleae Corylifoliae, Radix Scrophulariae Ningpoensis, Radix Platycodi. In some embodiments, the senosuppressor comprises a mixture of Stone CP, Jiuyuan, Tushi, Digupi, Wuweizi, and Chuanqiong. In some embodiments, the senosuppressor comprises a mixture of Stone CP, Jiuyuan, Turtle back, and Dragon Bone. In some embodiments, the senosuppressor comprises a mixture of Ginsen, Jiuyuan, Shengdi, Maidong, Tiandong, Danggui, Wuweizi, Pozi, Zhaoren, Xuansen, Fuling, and Jigeng. In some embodiments, the disease associated with the accumulation of senescent cells is an age-related disease. In some embodiments, the age-related disease is selected from the group consisting of neurodegenerative disease, ocular disease, cardiovascular disease, pulmonary disease, inflammatory disease, and a metabolic disease. In some embodiments, the neurodegenerative disease is Alzheimer's Disease and Parkinson's Disease. In some embodiments, the ocular disease is cataracts, macular degeneration, or glaucoma. In some embodiments, the cardiovascular disease is atherosclerosis or hypertension. In some embodiments, the pulmonary disease is idiopathic pulmonary fibrosis or chronic obstructive pulmonary fibrosis (COPD). In some embodiments, the inflammatory disease is osteoarthritis. In some embodiments, the metabolic disease is type 2 diabetes, obesity, or fat dysfunction. In some embodiments, the disease associated with the accumulation of senescent cells is chemotherapy-induced cognitive impairment. In some embodiments, the administering of the senosuppressor results in a reduction in expression of one or more senescence-associated secretory phenotype (SASP) factors relative to the reference sample. In some embodiments, the SASP factor is a growth arrest SASP factor. In some embodiments, the growth arrest SASP factor is selected from the group consisting of TGF-β, IL-6, and IGFβ3. In some embodiments, the SASP factor is an inflammatory SASP factor. In some embodiments, the inflammatory SASP factor is selected from the group consisting of IL-1α, IL-1β, IL-8, and IFN-γ. In some embodiments, the SASP factor is a stemness SASP factor. In some embodiments, the stemness SASP factor is GM-CSE. In some embodiments, the SASP factor is a remodeling of extracellular space SASP factor. In some embodiments, the remodeling of extracellular space SASP factor is selected from the group consisting of MMP-1, MMP-3, MMP-10, MMP-12, MMP-13, and MMP-14. In some embodiments, the quiescence-inducing compound is a CDK4/6 inhibitor. In some embodiments, the CDK4/6 inhibitor is PD0332991. In some embodiments, the quiescence-inducing compound is doxorubicin. In some embodiments, the quiescence-inducing compound causes a cancer cell to transition to quiescence. In some embodiments, the ATRX foci per cell are detected by immunofluorescence. In some embodiments, the ATRX foci per cell are detected by an ATRX-specific antibody. In some embodiments, the ATRX-specific antibody recognizes ATRX protein at the C-terminal domain. In some embodiments, the mean number or total number of ATRX foci per cell is detected at least 1 day, at least 2 days, at least 3 days, at least 4 days, at least 5 days, at least 6 days, or at least 7 days after administering the quiescence-inducing compound to the subject.

Disclosed herein are methods of treating a subject having a retroviral infection comprising administering to the subject an anti-retroviral medicament; and administering to the subject a therapeutically effective amount of a senosuppressor. In some embodiments, the anti-retroviral medicament is a protease inhibitor. In some embodiments, the retroviral infection is human immunodeficiency virus. In some embodiments, the senosuppressor modulates a number of ATRX foci in a cell. In some embodiments, the number of ATRX foci in the cell is reduced following administering of the senosuppressor relative to a reference sample. In some embodiments, the number of ATRX foci in the cell is reduced at least 3-fold following administering the senosuppressor relative to the reference sample. In some embodiments, the cell is derived from the subject. In some embodiments, the senosuppressor comprises an extract from a plant species selected from the group consisting of Albizia julibrissin Durazz, Arisaema, Arnebia, Lithospermum, Atractylodes macrocephala, Cnidium monnieri, Dimocarpus longan Lour, Forsythia suspensa Juncus effuses, Lilium, Lophatherum gracile Brongn, Nelumbo nucifera Gaertn, Pinellia ternate, Polygonum multiflorum, and Tribulus terrestris. In some embodiments, the senosuppressor comprises an extract from a species selected from the group consisting of Radix Ginseng, Radix Astragali, Fructus Viticis, Radix Puerariae, Cortex Phellodendri, Rhizoma Cimicifugae, Radix Glycyrrhizae, Rhizoma Acori Graminei, Radix Polygalae Tenuifoliae, Sclerotium Poriae Cocos, Scierotium Pararadicis Poriae Cocos, Semen Cuscutae Chinensis, Cortex Radicis Lycii Chinensis, Fructus Schisandrae Chinensis, Rhizoma Ligustici Chuanxiong, Radix Rehmanniea, Fructus Corni Officinalis, Semen Zizyphi Spinosae, Plastrum Testudinis, Os Draconis, Radix Rehmanniae Glutinosae, Tuber Ophiopogonis Japonici, Tuber Asparagi Cochinensis, Radix Angelicae Sinensis, Fructus Psoraleae Corylifoliae, Radix Scrophulariae Ningpoensis, Radix Platycodi. In some embodiments, the senosuppressor comprises a mixture of Stone CP, Jiuyuan, Tushi, Digupi, Wuweizi, and Chuanqiong. In some embodiments, the senosuppressor comprises a mixture of Stone CP, Jiuyuan, Turtle back, and Dragon Bone. In some embodiments, the senosuppressor comprises a mixture of Ginsen, Jiuyuan, Shengdi, Maidong, Tiandong, Danggui, Wuweizi, Pozi, Zhaoren, Xuansen, Fuling, and Jigeng. In some embodiments, the disease associated with the accumulation of senescent cells is an age-related disease. In some embodiments, the age-related disease is selected from the group consisting of neurodegenerative disease, ocular disease, cardiovascular disease, pulmonary disease, inflammatory disease, and a metabolic disease. In some embodiments, the neurodegenerative disease is Alzheimer's Disease and Parkinson's Disease. In some embodiments, the ocular disease is cataracts, macular degeneration, or glaucoma. In some embodiments, the cardiovascular disease is atherosclerosis or hypertension. In some embodiments, the pulmonary disease is idiopathic pulmonary fibrosis or chronic obstructive pulmonary fibrosis (COPD). In some embodiments, the inflammatory disease is osteoarthritis. In some embodiments, the metabolic disease is type 2 diabetes, obesity, or fat dysfunction. In some embodiments, the disease associated with the accumulation of senescent cells is chemotherapy-induced cognitive impairment. In some embodiments, the administering of the senosuppressor results in a reduction in expression of one or more senescence-associated secretory phenotype (SASP) factors relative to the reference sample. In some embodiments, the SASP factor is a growth arrest SASP factor. In some embodiments, the growth arrest SASP factor is selected from the group consisting of TGF-β, IL-6, and IGFβ3. In some embodiments, the SASP factor is an inflammatory SASP factor. In some embodiments, the inflammatory SASP factor is selected from the group consisting of IL-1α, IL-1β, IL-8, and IFN-γ. In some embodiments, the SASP factor is a stemness SASP factor. In some embodiments, the stemness SASP factor is GM-CSE. In some embodiments, the SASP factor is a remodeling of extracellular space SASP factor. In some embodiments, the remodeling of extracellular space SASP factor is selected from the group consisting of MMP-1, MMP-3, MMP-10, MMP-12, MMP-13, and MMP-14. In some embodiments, the ATRX foci are detected by immunofluorescence. In some embodiments, the ATRX foci are detected by an ATRX-specific antibody. In some embodiments, the ATRX-specific antibody recognizes ATRX protein at the C-terminal domain. In some embodiments, the cell derived from the subject is contained in a sample wherein the ATRX foci are detected at least 1 day, at least 2 days, at least 3 days, at least 4 days, at least 5 days, at least 6 days, or at least 7 days following administering a quiescence-inducing compound to the sample.

Disclosed herein are methods for treating a neoplastic disease in a subject comprising: administering to the subject a therapeutically effective amount of a senostimulator that increases a mean number of ATRX foci per cell at least 3-fold in a sample derived from the subject after the subject was administered a quiescence-inducing compound relative to a mean number of ATRX foci per cell in a reference sample collected from the subject before the administering of the senostimulator and after the administering of the quiescence-inducing compound.

Also disclosed herein are methods for treating a neoplastic disease in a subject who has received a quiescence-inducing compound. In some cases, the subject has had a partial response to the quiescence-inducing compound. In some cases, a partial response may include a response followed by a relapse. In some cases, a method of treating a neoplastic disease in a subject who has received a quiescence-inducing compound comprises administering to the subject a therapeutically effective amount of a senostimulator that increases a number of ATRX foci per cell in a sample derived from the subject after the subject was administered the quiescence-inducing compound such that at least 30% of the cells have a number of ATRX foci that is at least 3 times higher than a mean number of ATRX foci per cell in a reference sample collected from the subject before the administering of the senostimulator and after the administering of the quiescence-inducing compound. In some cases, a method of treating a neoplastic disease in a subject comprises administering to the subject a therapeutically effective amount of a quiescence-inducing compound and a senostimulator such that at least 30% of the cells in a sample take from the subject after treatment have a number of ATRX foci that is at least 3 times higher than a mean number of ATRX foci per cell in a reference sample collected from the subject before the administering of the quiescence-inducing compound and the senostimulator.

In some embodiments, the neoplastic disease is a cancer. In some embodiments, the administering of the senostimulator results in an increase in expression of one or more senescence-associated secretory phenotype (SASP) factors relative to the reference sample. In some embodiments, the SASP factor is a growth arrest SASP factor. In some embodiments, the growth arrest SASP factor is selected from the group consisting of TGF-β3, IL-6, and IGFβ3. In some embodiments, the SASP factor is an inflammatory SASP factor. In some embodiments, the inflammatory SASP factor is selected from the group consisting of IL-1α, IL-1β, IL-8, and IFN-γ. In some embodiments, the SASP factor is a stemness SASP factor. In some embodiments, the stemness SASP factor is GM-CSE. In some embodiments, the SASP factor is a remodeling of extracellular space SASP factor. In some embodiments, the remodeling of extracellular space SASP factor is selected from the group consisting of MMP-1, MMP-3, MMP-10, MMP-12, MMP-13, and MMP-14. In some embodiments, the quiescence-inducing compound is a CDK4/6 inhibitor. In some embodiments, the CDK4/6 inhibitor is PD0332991. In some embodiments, the quiescence-inducing compound is doxorubicin. In some embodiments, the quiescence-inducing compound causes a cancer cell to transition to quiescence. In some embodiments, the ATRX foci per cell are detected by immunofluorescence. In some embodiments, the ATRX foci per cell are detected by an ATRX-specific antibody. In some embodiments, the ATRX-specific antibody recognizes ATRX protein at the C-terminal domain. In some embodiments, the mean number of ATRX foci per cell is detected at least 1 day, at least 2 days, at least 3 days, at least 4 days, at least 5 days, at least 6 days, or at least 7 days after administering the quiescence-inducing compound to the subject.

Disclosed here in are methods of treating a subject comprising administering to the subject a senostimulator treatment, wherein the subject has previously received a quiescence-inducing compound treatment and a first senostimulator treatment resulting in at least a 3-fold increase in the mean number of ATRX foci per cell in a sample derived from the subject relative to a mean number of ATRX foci per cell in a reference sample collected from the subject prior to the first senostimulator treatment but after administration of the quiescence-inducing compound to the subject. In some embodiments, the subject has one or more symptoms of a neoplastic disease. In some embodiments, the neoplastic disease is a cancer. In some embodiments, the senostimulator causes an increase in expression of one or more senescence-associated secretory phenotype (SASP) factors relative to a reference sample. In some embodiments, the SASP factor is a growth arrest SASP factor. In some embodiments, the growth arrest SASP factor is selected from the group consisting of TGF-β, IL-6, and IGFβ3. In some embodiments, the SASP factor is an inflammatory SASP factor. In some embodiments, the inflammatory SASP factor is selected from the group consisting of IL-1α, IL-1β, IL-8, and IFN-γ. In some embodiments, the SASP factor is a stemness SASP factor. In some embodiments, the stemness SASP factor is GM-CSE. In some embodiments, the SASP factor is a remodeling of extracellular space SASP factor. In some embodiments, the remodeling of extracellular space SASP factor is selected from the group consisting of MMP-1, MMP-3, MMP-10, MMP-12, MMP-13, and MMP-14. In some embodiments, the senostimulator causes an increase in senescence-associated beta-galactosidase relative to the reference sample. In some embodiments, the quiescence-inducing compound is CDK4/6 inhibitor. In some embodiments, the CDK4/6 inhibitor is PD0332991. In some embodiments, the quiescence-inducing compound is doxorubicin. In some embodiments, the quiescence-inducing compound causes a cancer cell to transition to quiescence. In some embodiments, the ATRX foci per cell are detected by immunofluorescence. In some embodiments, the ATRX foci per cell are detected by an ATRX-specific antibody. In some embodiments, the ATRX-specific antibody recognizes ATRX protein at the C-terminal domain. In some embodiments, the mean number of ATRX foci per cell is detected at least 1 day, at least 2 days, at least 3 days, at least 4 days, at least 5 days, at least 6 days, or at least 7 days after administering the quiescence-inducing compound to the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 is a schematic of the senescence after growth arrest pathway indicating the central role that Ras signaling plays in ATRX function during this process.

FIG. 2 illustrates the percentage of cells that incorporated BrdU at different timepoints during 8 days of treatment with palbociclib.

FIG. 3 illustrates the percentage of cells with a specific number of ATRX foci at different timepoints during the 8 days of treatment with palbociclib.

FIGS. 4A-4B provide representative images, at the same magnification, from untreated cycling cells and treated cells 8 days after addition of palbociclib. FIG. 4A shows a representative image of ATRX foci (green) in untreated cycling H1975 cells and FIG. 4B shows a representative image of ATRX foci in cells treated with palbociclib for 8 days.

FIG. 5 provides histograms illustrating the percent of cells with each specific number of ATRX foci after doxycyclin treated LS8817^{TetONFMDM2} cells (cycling) were treated with 0.2 μM palbociclib for 72 hours to induce quiescense (Q3) and then released from doxycycline for 72 hours to induce senescence after growth arrest (SAGA3-3).

FIGS. 6A-6B provide representative images of the ATRX foci in the cells of FIG. 5. The raw image, FIG. 6A, and a segmented image, FIG. 6B, are shown. In the segmented image in FIG. 6B each colored spot represents a unique focus identified by automated analysis.

FIG. 7 illustrates the accumulation of ATRX foci in LS8817^{TetONFMDM2} cells treated with doxycycline, doxycycline and palbociclib, or doxycycline, palbociclib and tipifarnib.

FIGS. 8A-8D show representative images of ATRX foci in cells treated with either 10 μg/ml doxycycline alone for 8 days (FIG. 8A), doxycycline for two days followed by three days of 10 μg/ml doxycycline and 0.2 μM palbociclib (FIG. 8B), doxycycline for two days followed by eight days of 10 μg/ml doxycycline and 0.2 μM palbociclib (FIG. 8C), or doxycycline for two days followed by three days of doxycycline and palbociclib followed by five days of doxycycline+palbociclib+0.1 μM tipifarnib (FIG. 8D). ATRX foci are shown in green, and DAPI staining in blue.

FIG. 9 shows the percentage of cells which are positive for SA-β-gal after treatment with doxycycline alone, tipifarnib alone, palbociclib alone, 3 or 8 days of doxycycline and palbociclib, or 3 days of doxycycline and palbociclib followed by 5 days of doxycycline, palbociclib and tipifarnib as described for FIGS. 8A-8D.

FIG. 10 illustrates SA-β-gal accumulation in a cell line that undergoes palbociclib arrest without senescence following treatment of the palbociclib treated cells for an additional 8 days with palbociclib and the indicated drugs.

FIGS. 11A-11C illustrate SA-β-gal staining in normal diploid fibroblasts (NDF) and fibroblasts (HGPS127) from a patient with Hutchinson-Gilford progeria syndrome (HGPS) a premature aging syndrome. The fibroblasts were cultured and treated with 0.5 μM calcitriol for the indicated number of days. FIG. 11A and FIG. 11B show representative images of SA-β-gal negative and positive cells respectively. FIG. 11C shows the percentage of cells which were positive for SA-β-gal at each timepoint in both the NDF and HGPS cells.

FIG. 12 illustrates nuclear area in normal diploid fibroblasts and fibroblasts from a patient with premature aging syndrome (HGPS127), as in FIGS. 11A-11C.

FIG. 13 illustrates the relationship between number of ATRX foci in a cell and the nuclear area of the cell, for cells treated as in FIG. 12. The horizontal line demarcates the 90th percentile in ATRX foci number, a stringent definition of the senescent state. The vertical line demarcates the 70th percentile of nuclear area. Cells in the upper right quadrant are senescent (ATRX^{high}NV^{large}). Cells in the lower right (ATRX^{low}NV^{large}) are moving along the path to senescence. Note the diminishment in the number of ATRX foci and the restoration of a normal nuclear size distribution of HGPS cells following calcitriol addition with time.

FIG. 14 illustrates different natural products, and the different mixtures of these which were used in Example 5.

FIG. 15 illustrates experimental design of an experiment to test the effect of aqueous extracts of herbal mixtures on palbociclib induced cellular senescence in LS8817 cells.

FIG. 16A illustrates the percentage of cells with different numbers of ATRX foci, after treatment with different aqueous extracts as described in FIG. 15. Black bars show untreated cycling cells, gray bars show palbociclib treated cells, and red bars show cells treated with palbociclib and an aqueous extract.

FIG. 16B illustrates the average number of ATRX foci per cell in untreated cells and cells treated with different concentrations of the aqueous extracts described in FIG. 15. The final concentrations of the extracts were produced by diluting different volumes of aqueous extract in 1 mL of media. The amount of μL of extract added are indicated in FIG. 16B by the number following the “E” in the x-axis labels. The double asterisk above the bar indicate that the data is equivalent to the amount used in FIG. 16A. The double carat above the bar indicates that there was an increased level of cell death at this dose of aqueous extract and thus the amount of ATRX foci/cell may not be representative of the activity.

FIG. 17 illustrates the percentage of cells positive for senescence associated beta-galactosidase activity in the indicated treatment groups.

FIG. 18 illustrates the percentage of the number of viable cells in palbociclib and extract conditions divided by the number of cell in palbociclib alone as a surrogate measure of viability. The data shown is representative of at least three different experiments for each mixture analyzed at multiple doses.

FIG. 19 illustrates experimental design of an experiment to test the effect of aqueous extracts of herbal mixtures on palbociclib induced cellular senescence in HGPS and NDF cells.

FIG. 20 illustrates the relationship between number of ATRX foci in a cell on the y-axis and the nuclear area of the cell on the x-axis. The horizontal line demarcates the 90^{th} percentile in ATRX foci number. Nuclear area was plotted on the x-axis. The vertical line demarcates the 70^{th} percentile of nuclear area. Cells in the upper right quadrant are senescent (ATRX^{hi}NV^{large}). The percentage of cells in the upper right quadrant is shown. NDF cells are on the left and HGPS on the right. The drug treatment is shown to the left of each pair of graphs.

FIG. 21 illustrates the percentage of the number of viable cells in each extract treated condition divided by the number of cells if left untreated as a surrogate measure of viability. The data shown is representative of at least three different experiments for each mixture analyzed at multiple doses and multiple times.

FIG. 22 provides a summary of the responses of LS8817 cells (inhibition of cellular senescence induced by palbociclib), HGPS cells and NDF cells treated with different aqueous extracts from the mixtures of FIG. 14. Toxicity in each cell as well as effect on senescence is shown. This data is the summary of at least two independent experiments for each mixture.

### DETAILED DESCRIPTION OF THE INVENTION

As average lifespans increase the burden of aging associated diseases and disorders also increases. Aging, and potentially accelerated aging in diseases such as progeria, is related to cellular and organismal stress. While the sensors and transducers may not be conserved, several outcomes including irreversible cell cycle exit, acquisition of a more apoptosis resistant phenotype, and adoption of an inflammation inducing secretory phenotype are well conserved. The appearance of senescent cells in prematurely aging skin has been reported to lead to age-related dermal and epidermal thickening and loss of collagen. Senescence in astrocytes may promote age-related neurodegeneration giving rise to cognitive impairment and contribute to Alzheimer's and Parkinson's diseases. Further, senescent chondrocytes are thought to play a role in osteoarthritis and senescent endothelial cells and smooth muscle cells may contribute to atherosclerosis. A causal link between senescence and aging was reported in a progeroid mouse model in which p16-positive senescence cells could be eliminated early in mouse life, and even later in life, and many age-related dysfunctions were delayed by the elimination of these senescent cells. Currently, senolytic treatments may eliminate beneficial senescent cells associated with stem cell reprogramming and wound repair. An alternative approach is to slow the development of senescent cells from quiescent cells.

Senescence is a state of stable cell cycle exit; senescent cells have withdrawn from the mitotic cycle and are refractory to signals that could stimulate their return. The replicative proficiency of cells that have exited the cell cycle has important consequences. For example, stem cell pools are actively maintained in quiescence—a non replicating state with the ability to return to the mitotic cycle. Losing these cells from quiescence to senescence depletes the stem cell pool and is believed to be a mechanism of aging.

Cellular senescence can be triggered by various stressors. The best understood molecular paradigms of cellular senescence are replicative senescence associated with telomere loss, oncogene induced senescence and Pten-loss induced senescence. Another form of senescence is therapy-induced senescence, a growth suppressive program activated by some cancer cells in response to cytostatic agents. Two features which may be present in senescent cells are senescence associated secretory program, (SASP), a cytokine expression profile leading to inflammation, and an increase in facultative heterochromatin known as the senescence-associated heterochromatic foci (SAHF). These features are believed to prevent the cells from returning to the cell cycle once the inducing stimuli are removed. While SAHFs have not been observed in all forms of senescent cells they are believed to be required for senescence in some contexts. In some cases, SAHFs may be required for formation of therapy induced senescent cells. One example of a protein which may be observed in SAHFs is the ATRX protein.

### ATRX

The alpha-thalassemialmental retardation syndrome X-linked protein (ATRX) is encoded by the atrx gene. ATRX is a SWI/SNF helicase/ATPase that can regulate gene expression via chromatin remodeling and may be associated with pericentric and telomeric heterochromatin. ATRX mutations are seen in the mental retardation syndrome α-thalassemia/MR, X-linked (ATRX syndrome).

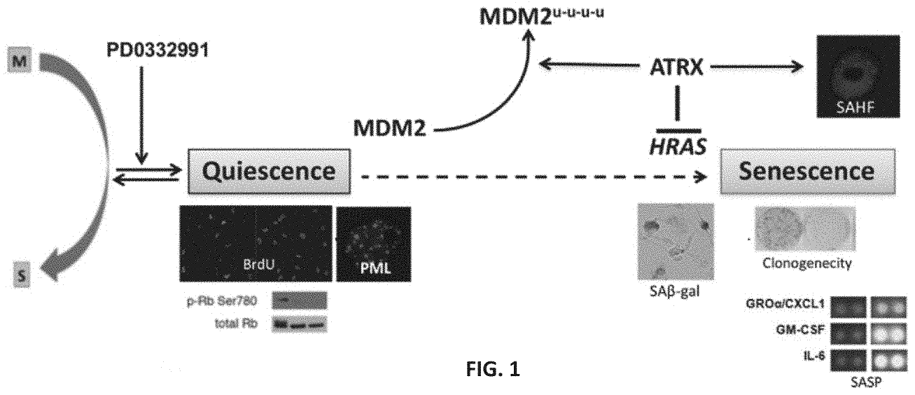

ATRX can interact with several proteins that are involved in senescence including PML bodies and macroH2A, and studies have shown that ATRX negatively regulates macroH2A (a facilitator of senescence-associated heterochromatic foci formation) incorporation into chromatin. ATRX has been shown to be involved in therapy-induced senescence. ATRX may accumulate in nuclear foci and may be required for therapy-induced senescence in multiple types of transformed cells exposed to either DNA damaging agents or CDK4 inhibitors. Mobilization of ATRX into foci may depend on the ability of ATRX to interact with H3K9me3 histone and HP1. These ATRX foci may form before other hallmarks of senescence appear. In some cases, eliminating ATRX in senescent cells destabilizes the senescence-associated heterochromatic foci. Additionally, ATRX binds to and suppresses expression from the HRAS locus; repression of HRAS is sufficient to promote the transition of quiescent cells into senescence and preventing repression blocks progression into senescence. FIG. 1 shows a schematic of the senescence after growth arrest pathway indicating the central role that Ras signaling plays in ATRX function during this process. Palbociclib (PD0332991) can induce G1 cell cycle exit into a non-cycling quiescent reversibly arrested cellular state marked by a reduction in bromodeoxyuridine (BrdU) incorporation and an increase in the number of PML nuclear bodies. These cells can then progress to an irreversibly arrested senescent state marked by the accumulation of senescence-associated beta-galactosidase (SA-β-gal), senescence associated heterochromatic foci marked by HP1γ (SAHF), long term clonogenic growth arrest when replated in the absence of palbociclib, and the elaboration of a transcriptional and secretion program of various cytokines and growth factors called the senescence associated secretory program or SASP. MDM2 ubiquitination and down-regulation is a key event in the process of senescence after growth arrest in cancer cells and enforcing MDM2 expression or preventing its downregulation will prevent palbociclib induced senescence, but the cells still accumulate in quiescence. ATRX may be necessary for senescence after growth arrest and it can participate in MDM2 down-regulation, suppression of HRAS transcription, and formation of the SAHF.

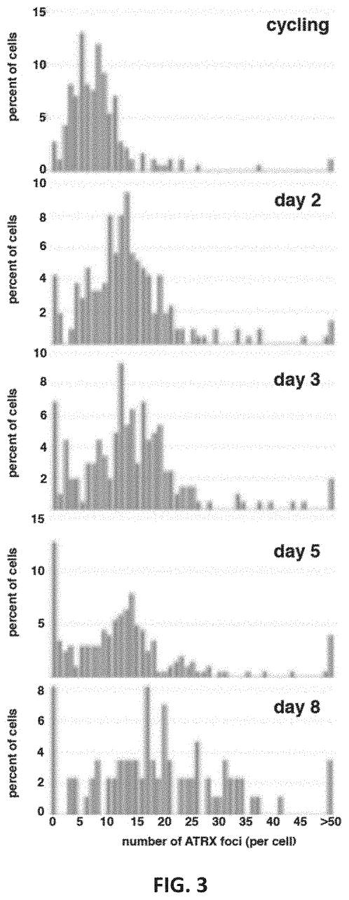

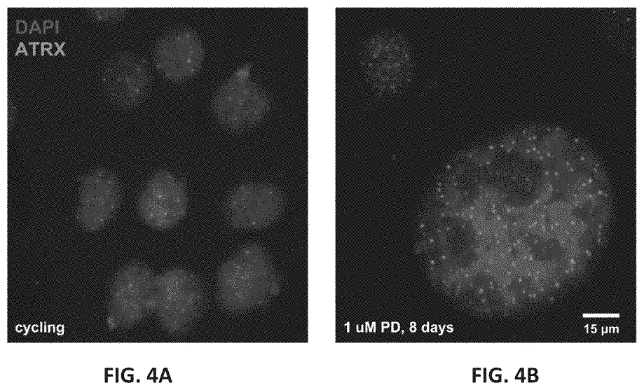

ATRX foci may be detected and quantified using any suitable method. For example, ATRX foci may be detected by immunofluorescence, as in FIGS. 4A and 4B. ATRX foci may be detected using an ATRX specific affinity reagent, such as an antibody or an aptamer. In some cases, an ATRX specific affinity reagent may recognize ATRX protein at the C-terminal domain. The number of ATRX foci per cell in a sample may be quantified manually or automatically using image processing software. FIGS. 3, 5, 7, 13, 16, and 20, for example, show the results of experiments in which ATRX foci were quantified after different treatments.

Senescent cells are typically cleared by the innate immune system which is recruited by SASP. One factor contributing to the increase in senescent cells during aging may be reduced clearance by the innate immune system.

### Methods of Treatment

### Senosuppressors

In some embodiments disclosed herein is a method of treating or delaying onset of a disease associated with the accumulation of senescent cells in a subject, the method comprising: administering to the subject a therapeutically effective amount of a senosuppressor that modulates a number of ATRX foci per cell.

In some cases, the number of ATRX foci per cell is a measurement of a total number of ATRX foci per cell in a sample derived from a subject after the subject was administered a senosuppressor relative to a total number of ATRX foci per cell in a reference sample collected from the subject before the administering of senosuppressor. In some cases, the number of ATRX foci per cell is a measurement of a total number of ATRX foci per cell in a sample derived from the subject after the subject was administered a quiescence-inducing compound relative to a total number of ATRX foci per cell in a reference sample collected from the subject before the administering of the senosuppressor and after the administering of the quiescence-inducing compound.

In some cases, the number of ATRX foci per cell is a measurement of a total number of ATRX foci per cell in cells treated with a senosuppressor of relative to a total number of ATRX foci per cell in cells that were not treated with a senosuppressor. In some cases, the number of ATRX foci per cell is a measurement of a total number of ATRX foci per cell in cells treated with both a senosuppressor and a quiescence-inducing compound relative to a total number of ATRX foci per cell in cells treated with just the quiescence-inducing compound. The cells may be cells of a subject in need of treatment, a primary cell culture, or an immortalized cell line or cancer cell line. In some cases, the cells may be genetically engineered to have desired properties.

In some cases, treatment with a senosuppressor may result in a decrease in a number of ATRX foci per cell in at least 10%, 15%, 20%, 25%, 30%, 35%, 40%, 45%, 50%, 55%, 60%, 65%, 70%, 75%, 80%, 85%, 90%, or 95% of cells in a treated sample as compared to an untreated sample.

In some embodiments, the number of ATRX foci per cell is a measurement of a mean number of ATRX foci per cell in a sample derived from the subject after the subject was administered a senosuppressor and a quiescence-inducing compound relative to a mean number of ATRX foci per cell in a reference sample collected from the subject before the administering of the senosuppressor and after the administering of the quiescence-inducing compound.

In some embodiments, the number of ATRX foci per cell is a measurement of a mean number of ATRX foci per cell in cells treated with a senosuppressor and a quiescence-inducing compound relative to a mean number of ATRX foci per cell cells treated with only the quiescence-inducing compound.

In some cases, treatment with a senosuppressor may result in a decrease in a mean number of ATRX foci per cell in a sample by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or 10 fold as compared to a mean number of ATRX foci per cell in an untreated sample.

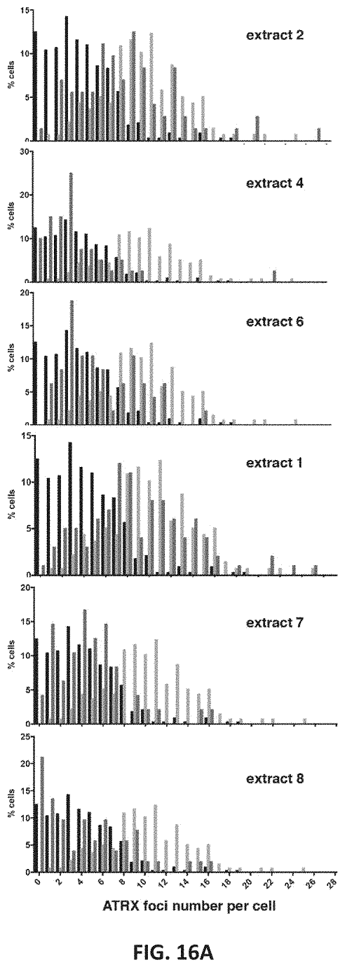

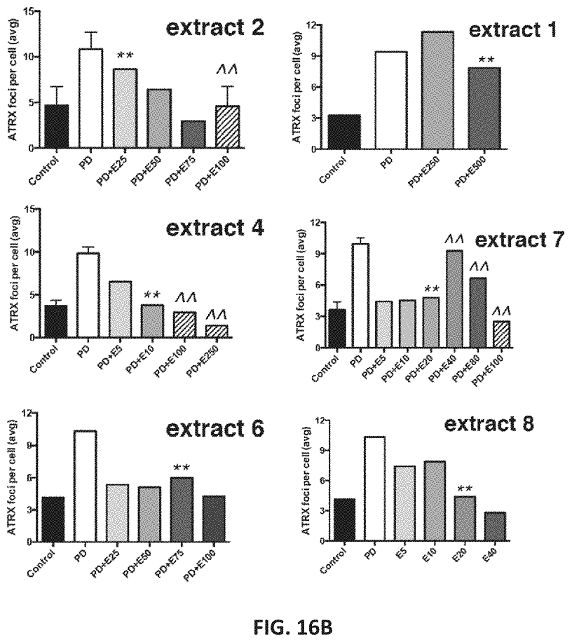

A senosuppressor may be a compound which prevents or inhibits the transition of a cell from a quiescent state to a senescent state. A senosuppressor may be a compound, or composition, which reduces or prevents the formation of senescent cells. In some cases, a senosuppressor may be a compound, or composition which reduces or prevents expression of a marker associated with senescent cells or a marker associated with the transition from quiescence to senescence. In some cases, a senosuppressor may be a compound, or composition which alters the localization of a marker associated with senescent cells or a marker associated with the transition from quiescence to senescence. For example, a senosuppressor may decrease the number of ATRX foci per cell as compared to similar cells without senosuppressor treatment. In some cases, a senosuppressor may reduce the average number of ATRX foci per cell in a treated sample as compared to a sample which was not treated with the senosuppressor. FIG. 16A shows the number of ATRX foci per cell in cells treated with a quiescence-inducing compound (palbociclib, gray bars) and then with different natural product extracts (red bars). Extracts 4, 6, 7, and 8 all shifted the curve to the right, indicating these extracts comprise senosuppressors. FIG. 16B shows the average number of ATRX foci per cell in untreated cells and cells treated with different aqueous extracts.

### Senescence Associated Diseases

Diseases or disorders which may be treated, prevented or delayed using a senosuppressor according to the methods described herein include all diseases and disorders associated with accumulation of senescent cells. Diseases and disorders associated with accumulation of senescent cells may be diseases and disorders of aging, or aging associated diseases and disorders. In some cases, the diseases and disorders associated with accumulation of senescent cells may be diseases or disorders of accelerated or premature aging such as progeria. Examples of diseases and disorders which may be associated with accumulation of senescent cells include, but are not limited to neurological diseases, neurodegenerative diseases, ocular diseases, cardiovascular diseases, pulmonary diseases, inflammatory diseases, and metabolic diseases. Neurological and neurodegenerative conditions include, but are not limited to, Parkinson's disease, Alzheimer's disease, dementia, amyotrophic lateral sclerosis (ALS), bulbar palsy, pseudobulbar palsy, primary lateral sclerosis, motor neuron dysfunction (MND), mild cognitive impairment (MCI), Huntington's disease, progressive muscular atrophy, lower motor neuron disease, spinal muscular atrophy (SMA), Werdnig-Hoffman Disease (SMA1), SMA2, Kugelberg-Welander Disease (SM3), Kennedy's disease, post-polio syndrome, hereditary spastic paraplegia, age-related memory decline, chemotherapy-induced cognitive impairment, and depression and mood disorders. Ocular diseases may include, but are not limited to, macular degeneration (wet and dry), glaucoma, vision loss, presbyopia, and cataracts. Examples of cardiovascular conditions, include, but are not limited to angina, arrhythmia, atherosclerosis, cardiomyopathy, congestive heart failure, coronary artery disease (CAD), carotid artery disease, endocarditis, coronary thrombosis, carotid thrombosis, myocardial infarction (MI), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease, peripheral artery disease (PAD), cardiac stress resistance, and stroke. Pulmonary conditions include, but are not limited to, idiopathic pulmonary fibrosis (IPF), chronic obstructive pulmonary disease (COPD), asthma, cystic fibrosis, bronchiectasis, emphysema, age-related loss of pulmonary function, and age-associated sleep apnea. Inflammatory conditions include, but are not limited to, osteoarthritis, osteoporosis, lupus, interstitial cystitis, scleroderma, alopecia, oral mucositis, rheumatoid arthritis, inflammatory bowel disease, kyphosis, herniated intervertebral disc, ulcerative colitis, Crohn's disease, ulcerative asthma, renal fibrosis including post-transplant renal fibrosis, liver fibrosis, pancreatic fibrosis, cardiac fibrosis, skin wound healing including diabetes related wound healing, and oral submucosa fibrosis. Metabolic conditions include, but are not limited to, diabetes (Type 1 or Type 2), metabolic syndrome, diabetic ulcers, obesity, fat dysfunction, renal dysfunction, nephrological pathology, and glomerular disease.

### Viral Infections

Diseases and disorders associated with accumulation of senescent cells may also include viral diseases, and side effects of viral therapies. In some cases, a viral disease may be a retroviral disease. Examples of retroviral diseases include, but are not limited to, human immunodeficiency virus (HIV, the virus that causes AIDS), human T-lymphotropic virus type 1 (HTLV-1) and human T-lymphotropic virus type 2 (HTLV-II). These diseases may be treated with anti-retroviral medicaments, which may cause cellular senescence. In some cases, the anti-retroviral medicament is a protease inhibitor. Examples of protease inhibitors that may be used as anti-retroviral therapies include lopinavir, indinavir, nelfinavir, amprenavir and ritonavir.

Provided herein are methods for treating, preventing, or delaying the onset of diseases or disorders associated with the accumulation of senescent cells. Treating may comprise reducing the number and/or severity of associated symptoms. In some cases, the methods provided herein may delay the onset of a disease. In some cases, onset of a disease may be delayed by about 3 months, 6 months, 9 months, or one year. In some cases, onset of a disease may be delayed by at least 1, 2, 3, 4, 5, 6, 7, 8, 9, 10, 11, 12, 13, 14, 15, 16, 17, 18, 19, or 20 years. In some cases, treating may comprise reducing the expression of a senescence marker. For example, treating may comprise reducing the expression of one or more senescence-associated secretory phenotype (SASP) factors relative to an untreated sample, or a reference sample. In some cases, treating may comprise reducing a number of senescence associated markers which are detectable in a treated sample, or subject, as compared to an untreated sample or subject. A SASP factor which shows reduced or undetectable expression after treatment according to the methods disclosed herein may be a growth arrest SASP, such as TGF-β3, IL-6, and/or IGFβ3. A SASP factor which shows reduced or undetectable expression after treatment according to the methods disclosed herein may be an inflammatory SASP, such as IL-1α, IL-1β, IL-8, and/or IFN-γ. A SASP factor which shows reduced or undetectable expression after treatment according to the methods disclosed herein may be a stemness SASP factor, such as GM-CSE. A SASP factor which shows reduced or undetectable expression after treatment according to the methods disclosed herein may be a remodeling of extracellular space SASP, such as MMP-1, MMP-3, MMP-10, MMP-12, MMP-13, and/or MMP-14.

A sample may be treated with both a quiescence-inducing compound and a senosuppressor. In some cases, a sample is treated with a quiescence-inducing compound prior to treatment with a senosuppressor. In some cases, a sample may be treated with a senosuppressor about 1, 2, 3, 4, 5, 6, 7, 8, 9, 10, or more than 10 days after treatment with a quiescence-inducing compound.

In some cases, treating with a senosuppressor according to the methods disclosed herein, may result in a reduction in the number of ATRX foci. In some cases, treating with a senosuppressor may result in a reduction in the number of ATRX foci per cell in a treated sample, as compared to an untreated sample.

### Senostimulators

In some embodiments disclosed herein is provided a method of treating a neoplastic disease in a subject comprising: administering to the subject a therapeutically effective amount of a senostimulator that increases a mean number of ATRX foci per cell at least 3-fold in a sample derived from the subject after the subject was administered a quiescence-inducing compound relative to a mean number of ATRX foci per cell in a reference sample collected from the subject before the administering of the senostimulator and after the administering of the quiescence-inducing compound.

In some cases, treating with a senostimulator according to the methods disclosed herein, may result in an increase in the number of ATRX foci. In some cases, treating with a senostimulator may result in an increase in the number of ATRX foci per cell in a treated sample, as compared to an untreated sample. In some cases, treatment with a senostimulator may result in an increase in a number of ATRX foci per cell in at least 10%, 15%, 20%, 25%, 30%, 35%, 40%, 45%, 50%, 55%, 60%, 65%, 70%, 75%, 80%, 85%, 90%, or 95% of cells in a treated sample as compared to an untreated sample. In some cases, treatment with a senostimulator may result in an increase in a mean number of ATRX foci per cell in a sample by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or 10 fold as compared to a mean number of ATRX foci per cell in an untreated sample. In some cases, treatment with a senostimulator may result in an increase in a number of ATRX foci per cell in a sample by at least 1.5 fold, 2 fold, 2.5 fold, 3 fold, 3.5 fold, 4 fold, 4.5 fold, or 5 fold, in at least 5%, 10%, 15%, 20%, 25%, 30%, 35%, 40%, 50%, or more than 50% of cells as compared to a number of ATRX foci per cell in an untreated sample. In some cases, treatment with a senostimulator may result in an increase in a total number of ATRX foci in a sample by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold or 10 fold as compared to a total number of ATRX foci in an untreated sample containing a comparable number of cells. In some cases, a number of ATRX foci is quantified before, or after about 1, 2, 3, 4, 5, 6, 7, 8, 9, 10 or more than 10 days after treatment with a senostimulator.

In some cases, a senostimulator may be a compound which promotes the transition of a quiescent cell to a senescent cell. A senostimulator may be a compound, or composition, which promotes the formation of senescent cells. In some cases, a senostimulator may be a compound, or composition, which increases a number, or an expression level, of a marker associated with senescent cells or a marker associated with the transition from quiescence to senescence. For example, a senostimulator may increase the number of ATRX foci per cell. In some cases, a senostimulator may increase the average number of ATRX foci per cell in a treated sample as compared to a sample which was not treated with the senosuppressor. In some cases, the senostimulator may cause an increase in senescence-associated beta-galactosidase relative to an untreated reference sample.

Provided herein are methods for treating neoplastic diseases. Treating may comprise reducing the number and/or severity of associated symptoms. In some cases, treating may comprise increasing the expression of a senescence marker. For example, treating may comprise increasing the expression of one or more senescence-associated secretory phenotype (SASP) factors relative to an untreated sample, or a reference sample. In some cases, treating may comprise increasing a number of senescence associated markers which are detectable in a treated sample, or subject, as compared to an untreated sample or subject. A SASP factor which shows increased expression after treatment according to the methods disclosed herein may be a growth arrest SASP, such as TGF-β, IL-6, and/or IGFβ3. A SASP factor which shows increased expression after treatment according to the methods disclosed herein may be an inflammatory SASP, such as IL-1α, IL-1β, IL-8, and/or IFN-γ. A SASP factor which shows increased expression after treatment according to the methods disclosed herein may be a stemness SASP factor, such as GM-CSE. A SASP factor which shows increased expression after treatment according to the methods disclosed herein may be a remodeling of extracellular space SASP, such as MMP-1, MMP-3, MMP-10, MMP-12, MMP-13, and/or MMP-14.

### Neoplastic Diseases

A neoplastic disease treated using a senostimulator may be any disease associated with an abnormal growth of cells. Examples of neoplastic diseases include cancer and benign tumors and growths. Examples of cancers include, but are not limited to, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

### Quiescence-Inducing Compounds

In some cases, a subject to be treated using the methods of the present disclosure may be undergoing treatment with a quiescence-inducing compound. The quiescence-inducing compound may be a therapeutic, for example a cancer therapeutic. In some cases, in order to observe an effect of a senosuppressor on the quiescence to senescence transition in cells it may be necessary to first treat the cells to induce quiescence. This may be done by treatment with a quiescence-inducing compound. In some cases, a quiescence inducing compound may cause a cancer cell to transition to quiescence. In some cases, a quiescence-inducing compound may be a senescence inducing compound. In some cases, a quiescence-inducing compound may be an antiretroviral protease inhibitor. In some cases, a quiescence inducing compound may be a CDK4/6 inhibitor such as PD0332991. In other cases, a quiescence-inducing compound may be doxorubicin. In some cases, a quiescence-inducing compound may be a cancer treatment. FIG. 10 shows the results of an experiment in which cells were primed with palbociclib to induce cell cycle exit without senescence, and then treated with several different candidate cancer therapeutics. Two of the candidate cancer therapeutics, tipifarnib and trametinib showed strong senescence-inducing activity.

### Assay

In some embodiments, a senomodulator as disclosed herein may be a compound which modulates the number of ATRX foci in cells. In some cases, the cells may be cells in a subject, cells in a tissue sample or biopsy taken from a subject, a cell line, or an engineered cell line. In some cases, the cells may be treated with a quiescence-inducing compound to induce a quiescent cell state.

In some embodiments, the cells may be cells which do undergo senescence when treated with the quiescence-inducing compound. In such cases, there may be a delay between the treatment of the cells with the quiescence-inducing compound and entry into senescence. In some cases, the cells may need to transition to a quiescent state before they are able to transition into a senescent state. Such cells may transition to a quiescent state and remain in that state for at least about 1, 2, 3, 4, 5, 6, 7, 8, 9, 10, 11, 12, 13, or 14 days.

In some embodiments, the cells may be cells which do not undergo senescence when treated with the quiescence-inducing compound. Such cells may transition to a quiescent state and remain in that state for at least 1, 2, 3, 4, 5, 6, 7, 8, 9, 10, 11, 12, 13, or 14 days. A method for identifying such a cell can comprise treating one or more cells with a senescence-inducing compound, e.g., a CDK4 inhibitor, and measuring accumulation of either SA-β-gal, senescence associated heterochromatic foci and/or elaboration of the senescence associated secretome. In certain non-limiting embodiments, a cell line that undergoes quiescence in response to treatment with the senescence-inducing compound does not increase expression of at least one marker, or at least two markers, or at least three markers of the senescent phenotype selected from the group consisting of SA-β-gal, senescence-associated heterochromatin foci and elaboration of the senescence-associated secretory program and/or does not increase the number of ATRX foci in the nucleus and/or exhibits stable or increased levels of MDM2 protein, relative to the level without treatment with the compound. In some cases, cells which do not undergo senescence when treated with a quiescence-inducing compound may undergo senescence when treated with a senostimulator.

In some cases, cells which do not undergo senescence when treated with a quiescence-inducing compound may be treated to induce senescence in the cells. In certain embodiments, senescence can be induced by the reduction in MDM2 expression in the cell. For example, and not by way of limitation, MDM2 expression can be reduced by the introduction of a modality that reduces MDM2 expression, e.g., a shRNA targeting MDM2 (i.e., shMDM2) or an siRNA targeting MDM2, into the cell. MDM2 expression may also be controlled by expressing MDM2 under the control of an inducible promoter, for example a Tet^{ON} promoter. In some cases, the reduction in MDM2 expression may occur simultaneously with the treatment of the cells with a senosuppressor. Alternatively, the cells can be treated with a senosuppressor at least one day, at least two days, at least three days, at least four days, at least five days, at least six days, at least seven days, at least eight days or more after the reduction in MDM2 expression. In certain non-limiting embodiments, the assay can further include determining the number of ATRX foci per cell following treatment with the senosuppressor. In certain embodiments, the assay can further include determining whether the one or more cells reenter the cell cycle by counting the total number of cells, e.g., by DAPI staining.

Senomodulators from Natural Products

A senomodulator may be a senosuppressor or a senostimulator. In some cases, the senomodulator, may comprise a natural product, or a natural product extract. For example, the senomodulator may comprise a plant extract, an animal extract, a fungal extract, or a bacterial extract. Examples of sources from which a senomodulator comprising extract may be produced include, but are not limited to, Radix Ginseng, Radix Astragali, Fructus Viticis, Radix Puerariae, Cortex Phellodendri, Rhizoma Cimicifugae, Radix Glycyrrhizae, Rhizoma Acori Graminei, Radix Polygalae Tenuifoliae, Sclerotium Poriae Cocos, Scierotium Pararadicis Poriae Cocos, Semen Cuscutae Chinensis, Cortex Radicis Lycii Chinensis, Fructus Schisandrae Chinensis, Rhizoma Ligustici Chuanxiong, Radix Rehmanniea, Fructus Corni Officinalis, Semen Zizyphi Spinosae, Plastrum Testudinis, Os Draconis, Radix Rehmanniae Glutinosae, Tuber Ophiopogonis Japonici, Tuber Asparagi Cochinensis, Radix Angelicae Sinensis, Fructus Psoraleae Corylifoliae, Radix Scrophulariae Ningpoensis, and Radix Platycodi. Examples of plants from which a senomodulator comprising extract may be produced include, but are not limited to, Albizia julibrissin Durazz, Arisaema, Arnebia, Lithospermum, Atractylodes macrocephala, Cnidium monnieri, Dimocarpus longan Lour, Forsythia suspensa Juncus effuses, Lilium, Lophatherum gracile Brongn, Nelumbo nucifera Gaertn, Pinellia ternate, Polygonum multiflorum, and Tribulus terrestris.

Further examples of natural products which may contain senomodulators include, but are not limited to Ginsen, Yellow Qi, Manjing, Gegen, Yellow Po, Rising Ma, Gan Weed, Stone CP, Jiuyuan, Fuling, Fushen, Tushi, Digupi, Wuweizi, Chuanqiong, Shudi, Shanzhu, Zhaoren, Turtle back, Dragon Bone, Shengdi, Maidong, Tiandong, Danggui, Pozi, Xuansen, and Jigeng. In some cases, a composition comprising a senomodulator may comprise extracts from one or more of Ginsen, Yellow Qi, Manjing, Gegen, Yellow Po, Rising Ma, Gan Weed, Stone CP, Jiuyuan, Fuling, Fushen, Tushi, Digupi, Wuweizi, Chuanqiong, Shudi, Shanzhu, Zhaoren, Turtle back, Dragon Bone, Shengdi, Maidong, Tiandong, Danggui, Pozi, Xuansen, and Jigeng.

In some cases, a composition comprising a senomodulator may comprise extracts from Ginsen, Yello Qi, Manjing, Gegen, Yellow Po, Rising Ma, and Gan weed. In some cases, a composition comprising a senomodulator may comprise extracts from Ginsen, Stone CP, Jiuyuan, Fuling, and Fushen. In some cases, a composition comprising a senomodulator may comprise extracts from Stone CP, Jiuyuan, Tushi, Digupi, Wuweizi, and Chuanqiong. In some cases, a composition comprising a senomodulator may comprise extracts from Ginsen, Stone CP, Jiuyuan, Shudi, Shanzhu, and Zhaoren. In some cases, a composition comprising a senomodulator may comprise extracts from Stone CP. Jiuyuan. Turtle back, and Dragon Bone. In some cases, a composition comprising a senomodulator may comprise extracts from Ginsen, Jiuyuan, Shengdi, Maidong, Tiandong, Danggui, Wuweizi, Pozi, Zhaoren, Xuansen, Fuling, and Jigeng. In some cases, a composition comprising a senomodulator may comprise extracts from Ginsen, Yellow Qi, Manjing, Gegen, Yellow Po, Rising Ma, and Gan Weed. In some cases, a composition comprising a senomodulator may comprise extracts from Ginsen, Stone CP, Jiuyuan, Fuling, and Fushen. In some cases, a composition comprising a senomodulator may comprise extracts from Ginsen, Stone CP, Jiuyuan, Shudi, Shanzu, and Zhaoren. In some cases, a composition comprising a senomodulator may comprise extracts from Ginsen, Jiuyuan, Fuling, Wuweizi, Zhaoren, Shengdi, Maidong, Tiandong, Dangqui, Pozi, Xuansen, Jigeng.

Ginsen may also be known by the Latin name: Radix Ginseng, the common names: Ginseng root, and Asian Ginseng root, and the Pinyin name: Ren Shen. Yellow Qi may also be known by the Latin name: Radix Astragali, the common names: Astragalus root and Milk Vetch root, the scientific name: Astragalus membranaceus or Astragalus mongholicus, and the Pinyin name: Huang qi. Manjing may also be known by the Latin name: Fructus Viticis, the common name: Vitex Fruit, and the Pinyin name: Man Jing Zi. Gegen may also be known by the Latin name: Radix Puerariae, the common name: Kudzu, and the Pinyin name: Ge Gen.

Yellow Po may also be known by the Latin name: Cortex Phellodendri, the common names: Phellodendron or Amur Cork tree Bark, and the Pinyin name: Huang Bai. Rising Ma may also be known by the Latin name: Rhizoma Cimicifugae, the common name: Black Cohosh Rhizome, and the Pinyin name: Sheng Ma. Gan Weed may also be known by the Latin name: Radix Glycyrrhizae, the common name: Licorice Root, and the Pinyin name: Gan Cao. Stone CP may also be known by the Latin name: Rhizoma Acori Graminei, the common name: Sweetflag Rhizome, and the Pinyin name: Shi Chang Pu. Jiuyuan may also be known by the Latin name: Radix Polygalae Tenuifoliae, the common name: Chinese Senega Root, and the Pinyin name: Yuan Zi. This herb often comes pre-treated and may appear darker when treated. Fuling may also be known by the Latin name: Sclerotium Poriae Cocos, the common name: China Root (type of wood fungus), and the Pinyin name: Fu Ling.

Fushen may also be known by the Latin name: Scierotium Pararadicis Poriae Cocos, the common name: Poria with host wood (same as Fu Ling but with attached wood), and the Pinyin name: Fu Shen. Tushi may also be known by the Latin name: Semen Cuscutae Chinensis, the common name: Chinese Dodder Seeds, and the Pinyin name: Tu Si Zi. Digupi may also be known by the Latin name: Cortex Radicis Lycii Chinensis, the common names: Cortex of Wolfberry Root, Lycium Bark, and the Pinyin name: Di Gu Pi. Wuweizi may also be known by the Latin name: Fructus Schisandrae Chinensis, the common name: Schisandra Fruit/seed, and the Pinyin name: Wu Wei Zi. In some cases, Wuweizi may reduce Liver enzyme (ALT) levels. Chuanqiong may also be known by the Latin name: Rhizoma Ligustici Chuanxiong, the common name: Szechuan Lovage Root, and the Pinyin name: Chuan Xiong. Shudi may also be known by the Latin name: Radix Rehmanniea, the common name: Chinese Foxglove root (pre-processed), and the Pinyin name: Shu Di Huang. Shanzhu may also be known by the Latin name: Fructus Corni Officinalis, the common name: Asiatic Cornelian Cherry Fruit, and the Pinyin name: Shan Zhu Yu. Zhaoren may also be known by the Latin name: Semen Zizyphi Spinosae, the common name: Sour Jujube seed, and the Pinyin name: Suan Zao Ren. Turtle back may also be known by the Latin name: Plastrum Testudinis, the common name: Fresh Water Turtle Shell, and the Pinyin name: Gui Ban.

Dragon Bone may also be known by the Latin name: Os Draconis, the common name: fossilized bones, and the Pinyin name: Long Gu. The main constituents in “dragon bone” and “dragon teeth” are calcium carbonate and calcium phosphate. Dragon Bone may also contain small amounts of iron, aluminum, silicate, sodium and potassium chloride, and traces of manganese, magnesium, titanium. In some cases, Dragon Bone may be paired with oyster shell or Mu Li.

Shengdi may also be known by the Latin name: Radix Rehmanniae Glutinosae, the common name: Chinese Foxglove Root (unprocessed), and the Pinyin name: Sheng Di Huang (“sheng” means raw or unprocessed). Maidong may also be known by the Latin name: Tuber Ophiopogonis Japonici, the common names: Ophiopogon Tuber, Creeping Lily-Turf Root and Dwarf Lily-Turf Root, and the Pinyin name: Mai Men Dong. Tiandong may also be known by the Latin name: Tuber Asparagi Cochinensis, the common name: Asparagus Root, and the Pinyin name: Tian Men Dong. Danggui may also be known by the Latin name: Radix Angelicae Sinensis, the common name: Chinese Angelica Root, and the Pinyin name: Dang Gui. Pozi may also be known by the Latin name: Fructus Psoraleae Corylifoliae, the common name: Psoralea Fruit, and the Pinyin name: Bu Gu Zi. Xuansen may also be known by the Latin name: Radix Scrophulariae Ningpoensis, the common name: Ningpo Figwort Root, and the Pinyin name: Xuan Shen. Jigeng may also be known by the Latin name: Radix Platycodi, the common name: Balloon Flower root, and the Pinyin name: Jie Geng.

### Subjects

The methods of this disclosure include treating a subject with a therapeutically effective amount of a senosuppressor. A subject of the present disclosure may be a human subject, or a non-human subject. Examples of non-human subjects include mice, rats, rabbits, monkeys, primates, cats, dogs, cattle, sheep and horses. A subject may be of any age. In some cases, a subject may be a child, an adult, or a geriatric adult. In some cases, a subject may be between about 20 and about 100 years of age, between about 30 and about 100 years of age, between about 45 and 80 years of age, or between about 35 and 70 years of age. In some cases, a subject is a child of about 1, 2, 3, 4, 5, 6, 7, 8, 9, 10, 11, 12, 13, 14, 15, 16, or 17 years of age.

In some cases, a subject treated according to the methods of this disclosure may be a subject who has previously received treatment with a senescence-inducing compound, or a senescence-inducing therapy. For example, a subject treated according to the methods of this disclosure may be a subject who has received a cancer treatment. In some cases, a subject treated according to the methods of this disclosure may be a subject who has received an antiretroviral protease inhibitor.

In some cases, a subject treated according to the methods of this disclosure may be a subject with an aging related disease. In some cases, a subject treated according to the methods of this disclosure may be an aging or elderly subject who does not yet show signs or symptoms of an aging related disease or disorder. In some cases, a subject treated according to the methods of this disclosure may be a subject with an accelerated aging disease or disorder, for example progeria. In some cases, a subject treated according to the methods of this disclosure may be a subject who has been treated, or is about to be treated, with a therapy known to be capable of inducing senescence.

In some cases, a subject treated according to the methods of this disclosure may be a subject with a neoplastic disease, for example a cancer patient.

### EXAMPLES

### Example 1

### Senescence in an In Vitro Cell Assay

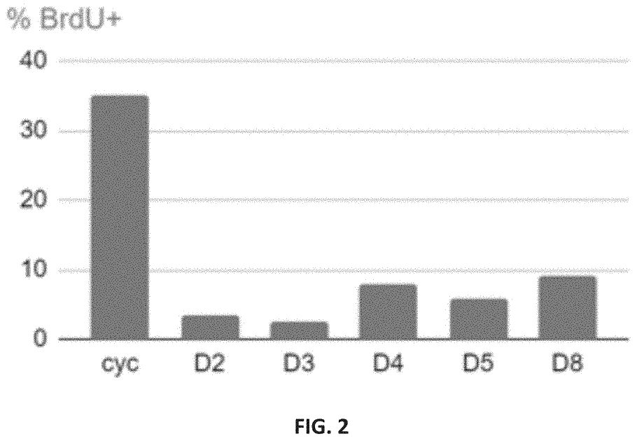

A lung carcinoma cell line (H1975) was used to assess palbociclib induced senescence. H1975 cells were treated with 1 μM palbociclib for up to 8 days to induce senescence. Cycling cells (cyc) were treated with vehicle alone for 8 days. At the end of the treatment period 20 μM bromodeoxyuridine (BrdU) was added to the media and the cells incubated an additional 90 minutes. Cells were then fixed and BrdU incorporation determined by immunohistochemistry. This data is representative of at least three independent experiments in which at least 100 cells were counted. FIG. 2 shows the percentage of cells that incorporated BrdU at each timepoint during the treatment. Cells at all the treated timepoints showed a lower rate of proliferation (indicated by a lower percentage of cells with BrdU incorporation) than untreated cells.

H1975 cells were treated with 1 μM palbociclib for 2-8 days. Cycling cells (cyc) were treated with vehicle alone for 8 days. After the indicated treatment times the cells were fixed and ATRX foci were detected by immunofluorescence. As seen in FIG. 3, which shows the percentages of cells with specific numbers of nuclear ATRX foci, the cells treated with palbociclib have higher numbers of nuclear ATRX foci. The number of nuclear ATRX foci is indicated on the x-axis and the percent of cells with that number is on the y-axis. Graphs were aligned to demonstrate the rightward shift of the peak. This data is representative of at least three independent experiments in which at least 100 cells were counted.

FIG. 4A shows a representative image of ATRX foci (green) in untreated cycling H1975 cells and FIG. 4B shows a representative image of ATRX foci in cells treated with palbociclib for 8 days. Note in FIG. 4B that the pre-senescent cells that are reversibly arrested can be distinguished from irreversibly arrested senescent cells by DAPI nuclear area (blue) even though the number of ATRX foci (green) are similar. The results in FIGS. 2-4 are consistent with palbociclib inducing senescence and show that the senescent cells have greater numbers of nuclear ATRX foci.

### Example 2

### Imaging and Quantitation of ATRX Foci Numbers for High Content Screening

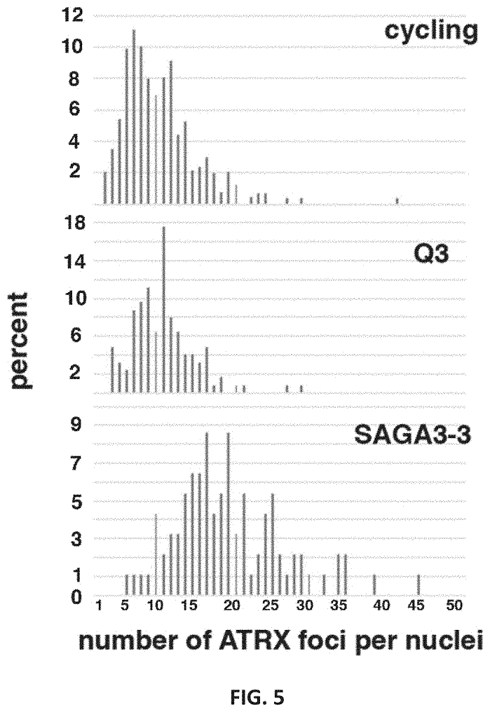

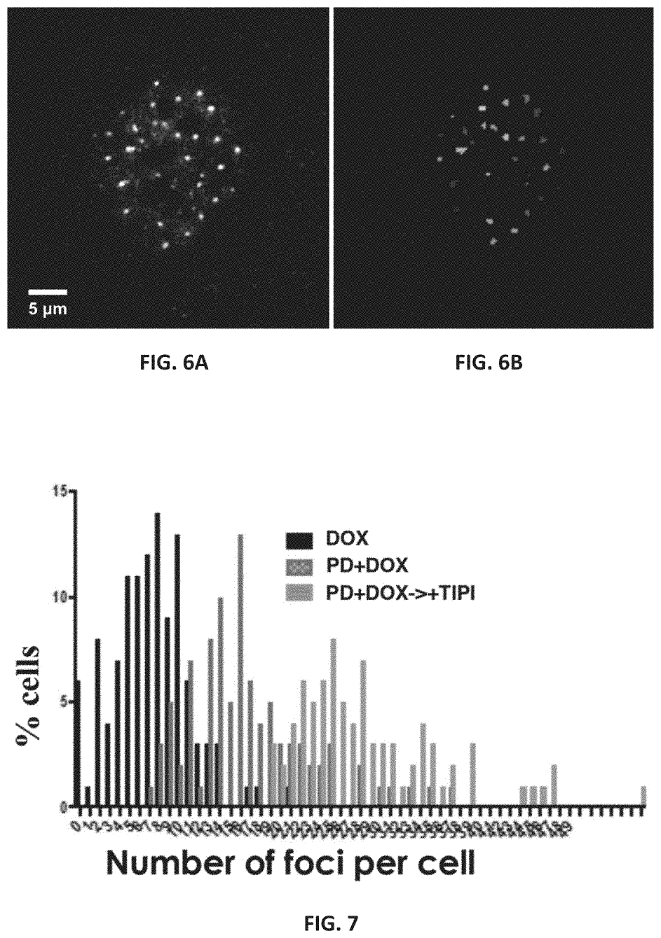

This study made use of a LS8817 sarcoma cell line which expresses FLAG-tagged Mouse double minute 2 homolog (MDM2) protein under the control of a tetracycline^{ON} promoter (LS8817^{TetONFMDM2}). When cultured with doxycycline these cells express FLAG-tagged MDM2 which prevents the cells from becoming senescent, even after treatment with palbociclib. These cells were then treated in the following manner. Cycling cells were untreated. Q3 cells were pre-treated with doxycycline for two days and then with 10 μg/ml doxycycline and 0.2 μM palbociclib for an additional three days to induce quiescence. SAGA3-3 cells were pre-treated with doxycycline for two days and then with doxycycline and palbociclib for an additional three days, and then doxycycline was removed and palbociclib alone continued for three days to induce senescence after growth arrest. The percentage of cells with a specific number of ATRX foci is plotted in FIG. 5, showing that the number of ATRX foci per nuclei does not change in the quiescent cells but is increased in the senescent after growth arrest cells. This is a representative example of at least three independent experiments for each condition. FIG. 6 shows a representative image of an automated ATRX foci count in a SAGA3-3 cell generated as described for FIG. 5. The raw image, FIG. 6A, and a segmented image, FIG. 6B, are shown. In the segmented image in FIG. 6B each colored spot represents a unique focus identified by automated analysis.

### Example 3

### The effect of Sequential Combination of Palbociclib with Other Drugs Targeting Various Signaling Pathways

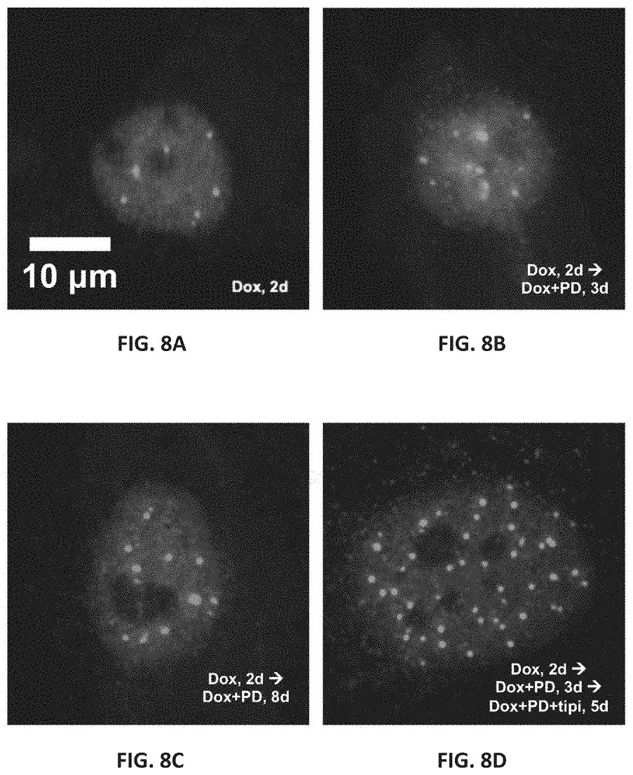

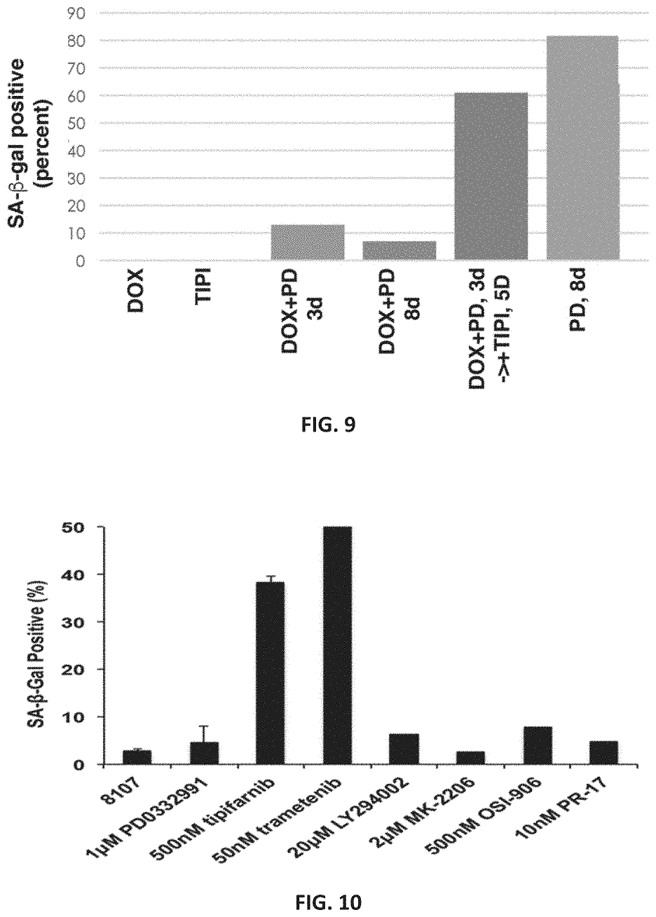

The ability of tipifarnib to induce senescence was confirmed in the LS8817^{TetONFMDM2} cells used in Example 2. LS8817^{TetONFMDM2} cells were treated with either doxycycline alone for 8 days (DOX, black), doxycycline for three days followed by two days of 10 μg/ml doxycycline and 0.2 μM palbociclib (PD+DOX, Brown), or doxycycline for two days followed by three days of doxycycline and palbociclib followed by five days of doxycycline+palbociclib+0.1 μM tipifarnib (PD+DOX->tipi, green). ATRX foci and nuclear size were evaluated as describe previously. As seen in FIG. 7 the cells treated with tipifarnib have an increased number of ATRX foci per cell. FIG. 8 shows representative images of ATRX foci in cells treated with either doxycycline alone for 8 days (FIG. 8A), doxycycline for three days followed by two days of 10 μg/ml doxycycline and 0.2 μM palbociclib (FIG. 8B), doxycycline for three days followed by five days of 10 μg/ml doxycycline and 0.2 μM palbociclib (FIG. 8C), or doxycycline for two days followed by three days of doxycycline and palbociclib followed by five days of doxycycline+palbociclib+0.1 μM tipifarnib (FIG. 8D). ATRX foci are shown in green, and DAPI staining in blue. FIG. 9 shows the percentage of cells which are positive for SA-β-gal after treatment with doxycycline alone, tipifarnib alone, palbociclib alone, 3 or 8 days of doxycycline and palbociclib, or 3 days of doxycycline and palbociclib followed by 5 days of tipifarnib as described for FIG. 8. The cells treated with 3 days of doxycycline and palbociclib followed by 5 days of tipifarnib had SA-β-gal in about 60% of cells compared to the cells treated with doxycycline and palbociclib for 8 days which had SA-β-gal in less than 10% of cells. Treatment with tipifarnib caused a greater than 6-fold increase in the number of senescent cells. Treatment with palbociclib alone (without doxycycline) resulted in about 80% of the cells being SA-β-gal positive.

A liposarcoma cell line (8107) in which palbociclib induces cell cycle exit but not senescence was used to determine the effect of several candidate cancer drugs on senescence. The 8107 cells were either untreated (8107), treated with palbociclib (PD0332991) for 8 days, or treated with palbociclib for 3 days and the indicated drugs at the indicated doses for an additional 5 days. The data from at least three independent experiments is plotted and the mean and standard deviation are shown. Two of the drugs, tipifarnib and trametinib, were able to induce senescence in these cells as indicated by the increase in SA-β-gal accumulation seen in FIG. 10.

### Example 4

### ATRX Foci in Fibroblast Skin Like Culture

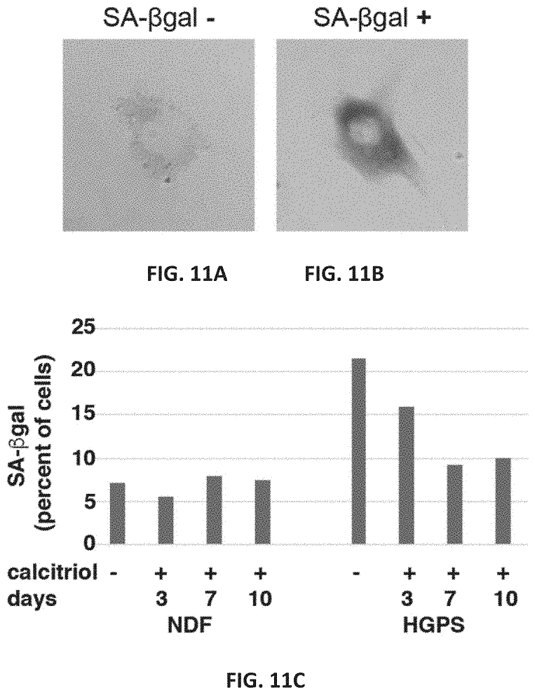

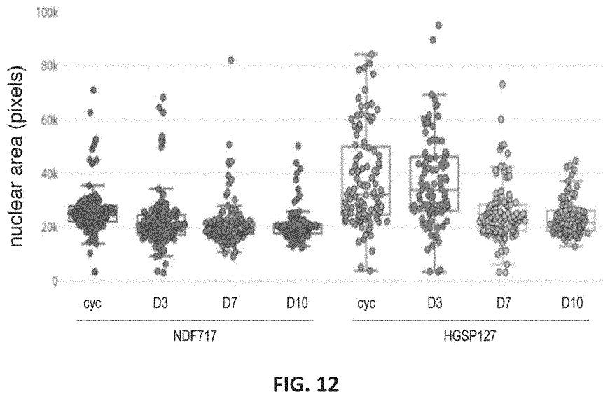

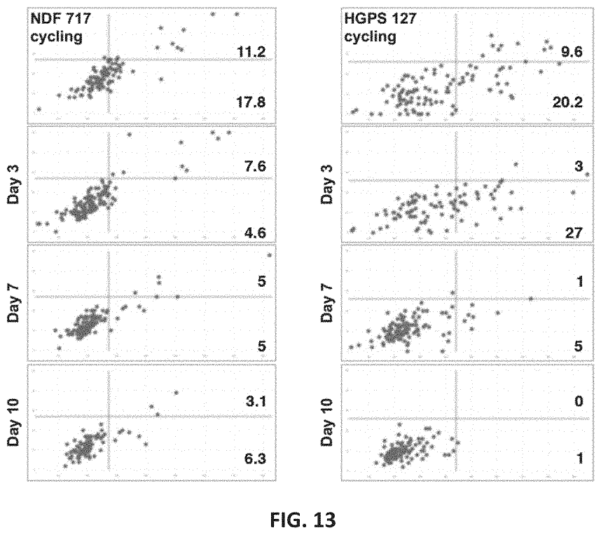

Normal diploid fibroblasts (NDF) and fibroblasts from a patient with premature aging syndrome (HGPS) were used to model human skin and to determine the effect of calcitriol on senescence in these cells. Calcitrol, a synthetic version of Vitamin D3, can reverse some symptoms of progeria. To assess the effect of calcitrol on senescence NDF and HGPS fibroblasts were cultured and treated with 0.5 μM calcitriol for up to 10 days. Cells were then stained for SA-β-gal. FIG. 11A and FIG. 11B show representative images of SA-β-gal negative and positive cells respectively. FIG. 11C shows the percentage of cells which were positive for SA-β-gal at each timepoint in both the NDF and HGPS cells. This experiment was repeated at least three times using different passages, a representative similarly passaged experiment is shown. FIG. 12 shows the nuclear area in the same cells determined by pixel density. FIG. 13 shows the relationship between nuclear area (x-axis) and the number of ATRX foci (y-axis). The horizontal line demarcates the 90th percentile in ATRX foci number, a stringent definition of the senescent state. The vertical line demarcates the 70th percentile of nuclear area. Cells in the upper right quadrant are senescent (ATRX^{high}NV^{large}). Cells in the lower right (ATRX^{low}NV^{large}) are moving along the path to senescence. Note the diminishment in the number of ATRX foci and the restoration of a normal nuclear size distribution of HGPS cells following calcitriol addition with time indicating that the calcitriol decreased the accumulation of senescent cells in these cultures. This experiment was repeated at least three times using different passages, a representative similarly passaged experiment is shown.

### Example 5

### Natural Product Extracts and their Effects on Cellular Senescence

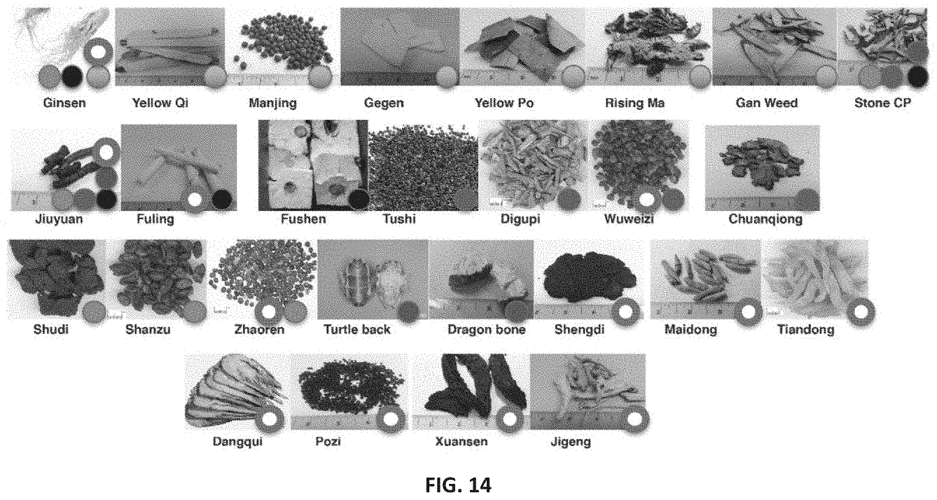

Several different natural product extracts were investigated for effects on senescence. FIG. 14 shows images of different natural products used to create the extracts. The symbols in FIG. 14 indicate different combinations of the natural products which were investigated for effects on senescence. Aqueous extracts were prepared by freezing the product mixtures in liquid nitrogen, grinding to a fine powder with a mortar and pestle on dry ice, and then boiling the powder with an equal volume of sterile distilled water for five minutes, slow cooling to room temperature, and repeating this cycle three times. After the third cooling period, extracts were clarified by centrifugation at 13,000×g for 20 minutes at 4° C. and then passed through a 0.22 μM filter and stored at −20° C. Activity was retained for at least three years.

Aqueous extract 1 was prepared from Ginsen, Yellow Qi, Manjing, Gegen, Yellow Po, Rising Ma, and Gan Weed. Aqueous extract 2 was prepared from Ginsen, Stone CP, Jiuyuan, Fuling, and Fushen. Aqueous extract 3 was prepared from Stone CP, Jiuyuan, Tushi, Digupi, Wuweizi, and Chuanqiong. Aqueous extract 4 was prepared from Ginsen, Stone CP, Jiuyuan, Shudi, Shanzu, and Zhaoren. Aqueous extract 5 was prepared from Stone CP, Jiuyuan, Turtle back, and Dragon bone. Aqueous extract 6 was prepared from Ginsen, Jiuyuan, Fuling, Wuweizi, Zhaoren, Shengdi, Maidong, Tiandong, Dangqui, Pozi, Xuansen, Jigeng.

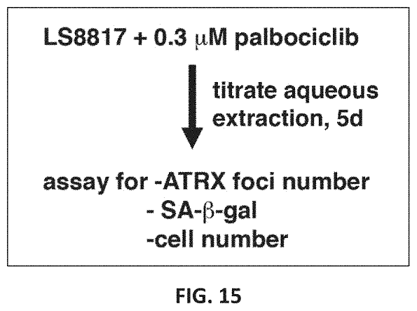

To test the effects of the natural product extracts they were applied to the LS8817 cell line described earlier. FIG. 15 shows the experimental design, briefly cells were treated with 0.3 μM palbociclib for 24 hours and then the media was replaced with 0.3 μM palbociclib and different amounts of the natural product extract for an additional 5 days. The percentage of cells with different numbers of ATRX foci are shown in FIG. 16A, black bars represent untreated cells, gray bars represent palbociclib treated cells, and red bars represent the cells treated with palbociclib and extract for seven days. These experiments were performed at least three times and representative graphs are shown. Extracts 4, 7, and 8 strongly inhibited palbociclib induced senescence in these cells. To further investigate the effect of the aqueous extracts a dose response experiment was conducted. LS8817 cells were treated with 0.3 μM palbociclib for 24 hours and then the media was replaced with 1 ml of media containing 0.3 μM palbociclib and different amounts of the natural product extract for an additional 5 days. The amount of μL of extract added are indicated in FIG. 16B by the number following the “E” in the x-axis labels. The average number of foci/cell was calculated at each different concentration of each aqueous extract and plotted. The double asterisk above the bar indicate that the data is equivalent to the amount used in FIG. 16A. The double carat above the bar indicates that there was increased levels of cell death at this dose of aqueous extract and thus the amount of ATRX foci/cell may not be representative of the activity.

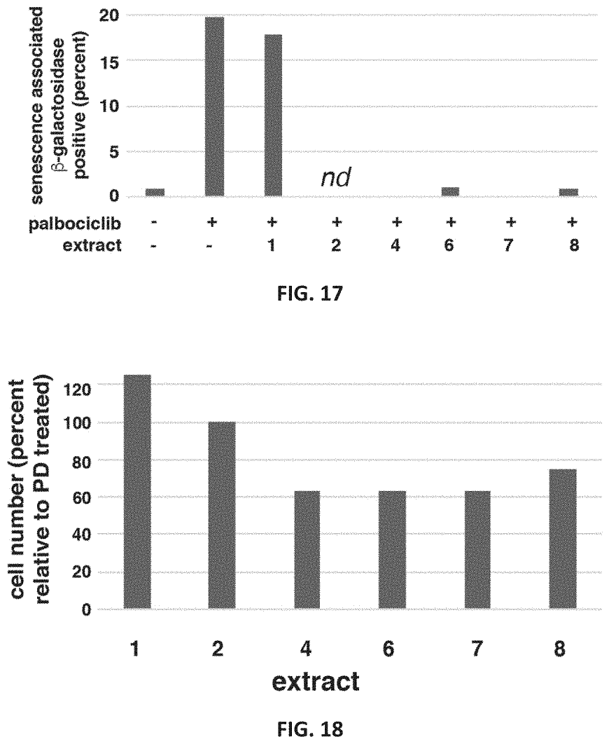

The percentage of cells with senescence associated beta-galactosidase activity was determined for each condition indicated and plotted in FIG. 17 (nd, not done). Extracts 4, 6, 7, and 8 all strongly suppressed the incidence of SA-β-gal. Extract 2 was not evaluated in this assay, data from one representative experiment is shown.

Cell number was used as a surrogate of viability. In FIG. 18 the y-axis is the percentage of viable cells in palbociclib and extract treated conditions divided by the number of cells in the palbociclib treated condition. The data shown is the average of at least three different experiments. Extracts 4, 6, 7, and 8 all show decreases in cell number, but the percentage of cells remains higher than 50%.

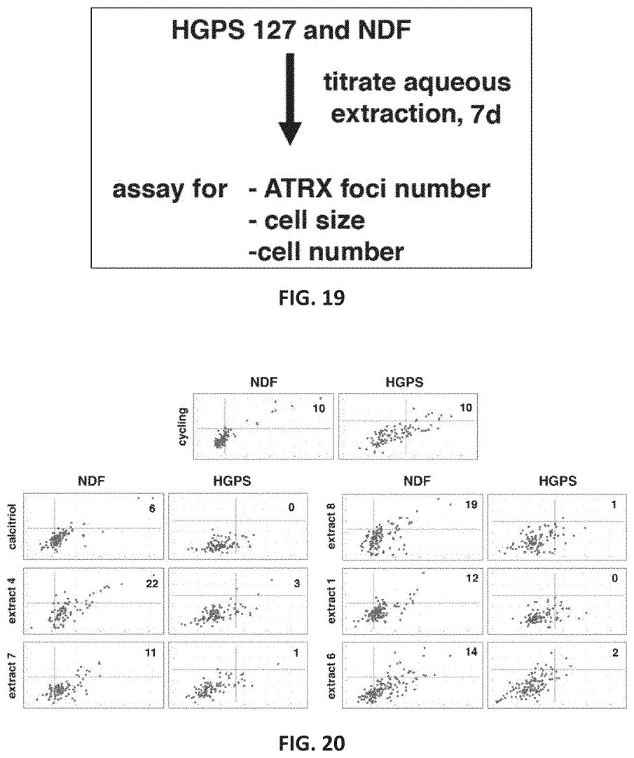

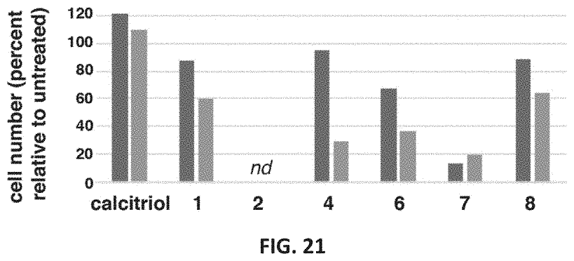

The natural extracts were also tested on fibroblast cells as described in Example 4. NDF and HGPS fibroblasts were treated with the natural product extracts for 7 days and then assessed for ATRX foci number, cell size and cell number, as indicated in the experimental design in FIG. 19. FIG. 20 shows quadrant plots of the number of ATRX foci (y-axis) vs the nuclear area (x-axis). The horizontal line demarcates the 90th percentile in ATRX foci number. The vertical line demarcates the 70th percentile of nuclear area. Cells in the upper right quadrant are senescent (ATRX^{high}NV^{large}). The percentage of cells in the upper right quadrant is shown. NDF cells are on the left and HGPS on the right. The drug treatment is shown to the left of each pair of graphs. Treatment with calcitriol reduced the percentage of senescent cells in the HGPS cells from about 10% to about 0%. Similar reductions in senescent cells were seen for all 5 extracts on the HGPS cells. Treatment of the NDF cells with the natural product extracts did not reduce the percentage of senescent cells, and in some cases increased the percentage of senescent cells. Cell number was used as a surrogate of viability to determine toxicity of the extracts. FIG. 21 shows the viable cells in extract treated conditions divided by the number if left untreated. Blue bars represent HGPS fibroblasts, and orange bars represent NDF cells. The data shown is representative of at least two different experiments for each mixture analyzed at multiple doses and multiple times.

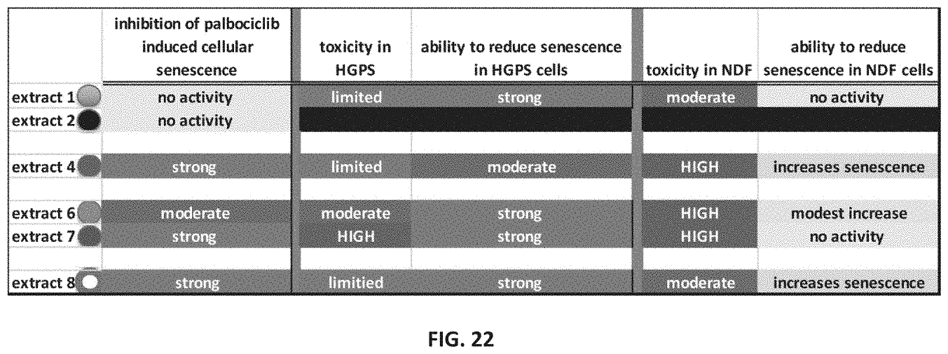

FIG. 22 provides a summary of the response in LS8817^{TetONFMDM2} cells (inhibition of cellular senescence induced by palbociclib) and in HGPS and NDF cells treated with different aqueous extracts from the mixtures. Toxicity in each cell, as well as the effect on senescence is shown. This data is the summary of at least two independent experiments for each mixture.

### Example 6

### Senosuppression in an In Vitro Cell Assay

A fibroblast or epithelial cell line will be plated into two different tissue culture plates. Cells in both plates will be treated with the CDK4/6 inhibitor PD0332991 for three days. Cells in the first dish will then be treated with a senosuppressor while the cells in the second dish will constitute the reference sample and will be treated with a vehicle control. After 3 days of treatment with the senosuppressor or vehicle control the cells will be fixed and stained for ATRX. The number of ATRX foci in each cell will be counted and used to calculate frequency histograms and a mean number of ATRX foci per cell. Cells in the first dish, which was treated with the senosuppressor, will have a lower mean number of ATRX foci per cell than cells in the reference sample.

### Example 7

### Senosuppression in a Mouse Aging Model

Ten one-year old C57B16 mice will be obtained and split into two groups of five mice each. Mice in one group will be administered a senosuppressor daily over a period of 12 months, while the mice in the other group will be administered a vehicle control. Each month a number of aging related phenotypes will be scored for each group. The mice receiving the senosuppressor will show delayed development of aging related phenotypes compared to the mice in the control group.

### Example 8

### A Clinical Trial Utilizing a Senosuppressor

Ten patients with mild symptoms of a disease associated with accumulation of senescent cells will be identified. The ten patients will be divided into two groups of five such that the two groups have comparable levels of the disease. The first group of five patients will be administered a daily dose of a therapeutically effective amount of a senosuppressor which modulates a number of ATRX foci per cell in an assay as in Example 1. The second group of five patients will be administered a daily dose of a placebo. Every month all ten patients will be clinically assessed for the number and severity of symptoms associated with the disease associated with accumulation of senescent cells. Patients in the first group, receiving the senosuppressor, will show fewer and less severe, symptoms than patients in the second group receiving the placebo.

### Example 9

### Senostimulation in an In Vitro Cell Assay

A fibroblast cell line will be plated into two different tissue culture plates. Cells in both plates will be treated with the CDK4/6 inhibitor PD0332991 for three days. Cells in the first dish will then be treated with a senostimulator while the cells in the second dish will constitute the reference sample and will be treated with a vehicle control. After 3 days of treatment with the senostimulator or vehicle control the cells will be fixed and stained for ATRX. The number of ATRX foci in each cell will be counted and used to calculate frequency histograms and a mean number of ATRX foci per cell. Cells in the first dish, which was treated with the senostimulator, will have a higher mean number of ATRX foci per cell than cells in the reference sample.

### Example 10

### Senostimulation in a Mouse Cancer Model

Ten C57B16 mice will be subcutaneously injected with B16 cancer cells. The mice will then be divided into two groups of five each. The mice in the first group will be administered a daily therapeutically effective dose of a senostimulator, while the mice in the second group will be administered daily placebo. The growth of the subcutaneous B16 cell tumors will be measured daily with calipers. The mice receiving the senostimulator will show slower growth of the tumor than the mice in the control group. In some mice receiving the senostimulator the B16 tumors may fail to develop.

### Example 11

### A Clinical Trial Utilizing a Senostimulator

Ten patients with sarcoma or carcinoma will be identified. The ten patients will be divided into two groups of five such that the two groups have comparable levels of the disease. All ten patients will receive the standard of care treatment. Additionally, the first group of five patients will be administered a daily dose of a therapeutically effective amount of a senostimulator which modulates a number of ATRX foci per cell in an assay as in Example 3. The second group of five patients will be administered a daily dose of a placebo. Every month all ten patients will be clinically assessed for disease progression and the number and severity of symptoms associated with the disease. Patients in the first group, receiving the senostimulator, will show slower disease progression, and fewer and less severe, symptoms than patients in the second group receiving the placebo.

### Example 12

### A Clinical Trial Utilizing a Senostimulator

Ten patients with lymphoma will be identified. The ten patients will be divided into two groups of five such that the two groups have comparable levels of the disease. All ten patients will receive the standard of care treatment for lymphoma. Additionally, the first group of five patients will be administered a daily dose of a therapeutically effective amount of a senostimulator which modulates a number of ATRX foci per cell in an assay as in Example 9. The second group of five patients will be administered a daily dose of a placebo. Every month all ten patients will be clinically assessed for disease progression and the number and severity of symptoms associated with the disease. Patients in the first group, receiving the senostimulator, will show slower disease progression, and fewer and less severe, symptoms than patients in the second group receiving the placebo.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating or delaying onset of a disease associated with the accumulation of senescent cells in a subject comprising:
administering to the subject a therapeutically effective amount of a senosuppressor that modulates a number of ATRX foci per cell.

2. The method of claim 1, wherein the number of ATRX foci per cell is a measurement of a total number of ATRX foci per cell in a sample derived from the subject after the subject was administered a quiescence-inducing compound relative to a total number of ATRX foci per cell in a reference sample collected from the subject before the administering of the senosuppressor and after the administering of the quiescence-inducing compound.

3. The method of claim 2, wherein the senosuppressor reduces the total number of ATRX foci per cell in at least 30% of cells in the sample derived from the subject relative to the reference sample.

4. The method of claim 2, wherein the total number of ATRX foci per cell in a sample derived from the subject is reduced at least 3-fold relative to the reference sample.

5. The method of claim 1, wherein the number of ATRX foci per cell is a measurement of a mean number of ATRX foci per cell in a sample derived from the subject after the subject was administered a quiescence-inducing compound relative to a mean number of ATRX foci per cell in a reference sample collected from the subject before the administering of the senosuppressor and after the administering of the quiescence-inducing compound.

6. The method of claim 5, wherein 30% of cells in the sample derived from the subject have a total number of ATRX foci per cell that is below the mean number of ATRX foci per cell in the reference sample.

7. The method of claim 5, wherein the mean number of ATRX foci per cell in the sample derived from the subject is reduced at least 3-fold relative to the reference sample.

8. The method of any one of claims 1-7, wherein the senosuppressor comprises an extract from a plant species selected from the group consisting of Albizia julibrissin Durazz, Arisaema, Arnebia, Lithospermum, Atractylodes macrocephala, Cnidium monnieri, Dimocarpus longan Lour, Forsythia suspensa Juncus effuses, Lilium, Lophatherum gracile Brongn, Nelumbo nucifera Gaertn, Pinellia ternate, Polygonum multiflorum, and Tribulus terrestris.

9. The method of any one of claims 1-7, wherein the senosuppressor comprises an extract from a species selected from the group consisting of Radix Ginseng, Radix Astragali, Fructus Viticis, Radix Puerariae, Cortex Phellodendri, Rhizoma Cimicifugae, Radix Glycyrrhizae, Rhizoma Acori Graminei, Radix Polygalae Tenuifoliae, Sclerotium Poriae Cocos, Scierotium Pararadicis Poriae Cocos, Semen Cuscutae Chinensis, Cortex Radicis Lycii Chinensis, Fructus Schisandrae Chinensis, Rhizoma Ligustici Chuanxiong, Radix Rehmanniea, Fructus Corni Officinalis, Semen Zizyphi Spinosae, Plastrum Testudinis, Os Draconis, Radix Rehmanniae Glutinosae, Tuber Ophiopogonis Japonici, Tuber Asparagi Cochinensis, Radix Angelicae Sinensis, Fructus Psoraleae Corylifoliae, Radix Scrophulariae Ningpoensis, Radix Platycodi.

10. The method of any one of claims 1-7, wherein the senosuppressor comprises a mixture of Stone CP, Jiuyuan, Tushi, Digupi, Wuweizi, and Chuanqiong.

11. The method of any one of claims 1-7, wherein the senosuppressor comprises a mixture of Stone CP, Jiuyuan, Turtle back, and Dragon Bone.

12. The method of any one of claims 1-7, wherein the senosuppressor comprises a mixture of Ginsen, Jiuyuan, Shengdi, Maidong, Tiandong, Danggui, Wuweizi, Pozi, Zhaoren, Xuansen, Fuling, and Jigeng.

13. The method of any one of claims 1-12 wherein the disease associated with the accumulation of senescent cells is an age-related disease.

14. The method of claim 13, wherein the age-related disease is selected from the group consisting of neurodegenerative disease, ocular disease, cardiovascular disease, pulmonary disease, inflammatory disease, and a metabolic disease.

15. The method of claim 14, wherein the neurodegenerative disease is Alzheimer's Disease and Parkinson's Disease.

16. The method of claim 14, wherein the ocular disease is cataracts, macular degeneration, or glaucoma.

17. The method of claim 14, wherein the cardiovascular disease is atherosclerosis or hypertension.

18. The method of claim 14, wherein the pulmonary disease is idiopathic pulmonary fibrosis or chronic obstructive pulmonary fibrosis (COPD).

19. The method of claim 14, wherein the inflammatory disease is osteoarthritis.

20. The method of claim 14, wherein the metabolic disease is type 2 diabetes, obesity, or fat dysfunction.

21. The method of any one of claims 1-12 wherein the disease associated with the accumulation of senescent cells is chemotherapy-induced cognitive impairment.

22. The method of any one of claims 1-21, wherein the administering of the senosuppressor results in a reduction in expression of one or more senescence-associated secretory phenotype (SASP) factors relative to the reference sample.

23. The method of claim 22, wherein the SASP factor is a growth arrest SASP factor.

24. The method of claim 23, wherein the growth arrest SASP factor is selected from the group consisting of TGF-β, IL-6, and IGFβ3.

25. The method of claim 22, wherein the SASP factor is an inflammatory SASP factor.

26. The method of claim 25, wherein the inflammatory SASP factor is selected from the group consisting of IL-1α, IL-1β, IL-8, and IFN-γ.

27. The method of claim 22, wherein the SASP factor is a stemness SASP factor.

28. The method of claim 27, wherein the stemness SASP factor is GM-CSE.

29. The method of claim 22, wherein the SASP factor is a remodeling of extracellular space SASP factor.

30. The method of claim 29, wherein the remodeling of extracellular space SASP factor is selected from the group consisting of MMP-1, MMP-3, MMP-10, MMP-12, MMP-13, and MMP-14.

31. The method of any one of claims 1-30, wherein the quiescence-inducing compound is a CDK4/6 inhibitor.

32. The method of claim 31, wherein the CDK4/6 inhibitor is PD0332991.

33. The method of any one of claims 1-32, wherein the quiescence-inducing compound is doxorubicin.

34. The method of any one of claims 1-33, wherein the quiescence-inducing compound causes a cancer cell to transition to quiescence.

35. The method of any one of claims 1-34, wherein the ATRX foci per cell are detected by immunofluorescence.

36. The method of claim 35, wherein the ATRX foci per cell are detected by an ATRX-specific antibody.

37. The method of claim 36, wherein the ATRX-specific antibody recognizes ATRX protein at the C-terminal domain.

38. The method of any one of claims 2-37, wherein the mean number or total number of ATRX foci per cell is detected at least 1 day, at least 2 days, at least 3 days, at least 4 days, at least 5 days, at least 6 days, or at least 7 days after administering the quiescence-inducing compound to the subject.

39. A method of treating a subject having a retroviral infection comprising:
administering to the subject an anti-retroviral medicament; and
administering to the subject a therapeutically effective amount of a senosuppressor.

40. The method of claim 39, wherein the anti-retroviral medicament is a protease inhibitor.

41. The method of claim 39, wherein the retroviral infection is human immunodeficiency virus.

42. The method of claim 39, wherein the senosuppressor modulates a number of ATRX foci in a cell.

43. The method of claim 42, wherein the number of ATRX foci in the cell is reduced following administering of the senosuppressor relative to a reference sample.

44. The method of claim 43, wherein the number of ATRX foci in the cell is reduced at least 3-fold following administering the senosuppressor relative to the reference sample.

45. The method of claim 43, wherein the cell is derived from the subject.

46. The method of any one of claims 39-45, wherein the senosuppressor comprises an extract from a plant species selected from the group consisting of Albizia julibrissin Durazz, Arisaema, Arnebia, Lithospermum, Atractylodes macrocephala, Cnidium monnieri, Dimocarpus longan Lour, Forsythia suspensa Juncus effuses, Lilium, Lophatherum gracile Brongn, Nelumbo nucifera Gaertn, Pinellia ternate, Polygonum multiflorum, and Tribulus terrestris.

47. The method of any one of claims 39-45, wherein the senosuppressor comprises an extract from a species selected from the group consisting of Radix Ginseng, Radix Astragali, Fructus Viticis, Radix Puerariae, Cortex Phellodendri, Rhizoma Cimicifugae, Radix Glycyrrhizae, Rhizoma Acori Graminei, Radix Polygalae Tenuifoliae, Sclerotium Poriae Cocos, Scierotium Pararadicis Poriae Cocos, Semen Cuscutae Chinensis, Cortex Radicis Lycii Chinensis, Fructus Schisandrae Chinensis, Rhizoma Ligustici Chuanxiong, Radix Rehmanniea, Fructus Corni Officinalis, Semen Zizyphi Spinosae, Plastrum Testudinis, Os Draconis, Radix Rehmanniae Glutinosae, Tuber Ophiopogonis Japonici, Tuber Asparagi Cochinensis, Radix Angelicae Sinensis, Fructus Psoraleae Corylifoliae, Radix Scrophulariae Ningpoensis, Radix Platycodi.

48. The method of any one of claims 39-45, wherein the senosuppressor comprises a mixture of Stone CP, Jiuyuan, Tushi, Digupi, Wuweizi, and Chuanqiong.

49. The method of any one of claims 39-45, wherein the senosuppressor comprises a mixture of Stone CP, Jiuyuan, Turtle back, and Dragon Bone.

50. The method of any one of claims 39-45, wherein the senosuppressor comprises a mixture of Ginsen, Jiuyuan, Shengdi, Maidong, Tiandong, Danggui, Wuweizi, Pozi, Zhaoren, Xuansen, Fuling, and Jigeng.

51. The method of any one of claims 39-50, wherein the disease associated with the accumulation of senescent cells is an age-related disease.

52. The method of claim 51, wherein the age-related disease is selected from the group consisting of neurodegenerative disease, ocular disease, cardiovascular disease, pulmonary disease, inflammatory disease, and a metabolic disease.

53. The method of claim 52, wherein the neurodegenerative disease is Alzheimer's Disease and Parkinson's Disease.

54. The method of claim 52, wherein the ocular disease is cataracts, macular degeneration, or glaucoma.

55. The method of claim 52, wherein the cardiovascular disease is atherosclerosis or hypertension.

56. The method of claim 52, wherein the pulmonary disease is idiopathic pulmonary fibrosis or chronic obstructive pulmonary fibrosis (COPD).

57. The method of claim 52, wherein the inflammatory disease is osteoarthritis.

58. The method of claim 52, wherein the metabolic disease is type 2 diabetes, obesity, or fat dysfunction.

59. The method of any one of claims 39-58, wherein the disease associated with the accumulation of senescent cells is chemotherapy-induced cognitive impairment.

60. The method of any one of claims 39-59, wherein the administering of the senosuppressor results in a reduction in expression of one or more senescence-associated secretory phenotype (SASP) factors relative to the reference sample.

61. The method of claim 60, wherein the SASP factor is a growth arrest SASP factor.

62. The method of claim 61, wherein the growth arrest SASP factor is selected from the group consisting of TGF-β, IL-6, and IGFβ3.

63. The method of claim 60, wherein the SASP factor is an inflammatory SASP factor.

64. The method of claim 63, wherein the inflammatory SASP factor is selected from the group consisting of IL-1α, IL-1β, IL-8, and IFN-γ.

65. The method of claim 60, wherein the SASP factor is a stemness SASP factor.

66. The method of claim 65, wherein the stemness SASP factor is GM-CSE.

67. The method of claim 60, wherein the SASP factor is a remodeling of extracellular space SASP factor.

68. The method of claim 67, wherein the remodeling of extracellular space SASP factor is selected from the group consisting of MMP-1, MMP-3, MMP-10, MMP-12, MMP-13, and MMP-14.

69. The method of any one of claims 39-68, wherein the ATRX foci are detected by immunofluorescence.

70. The method of claim 69, wherein the ATRX foci are detected by an ATRX-specific antibody.

71. The method of claim 70, wherein the ATRX-specific antibody recognizes ATRX protein at the C-terminal domain.

72. The method of claim 45, wherein the cell derived from the subject is contained in a sample wherein the ATRX foci are detected at least 1 day, at least 2 days, at least 3 days, at least 4 days, at least 5 days, at least 6 days, or at least 7 days following administering a quiescence-inducing compound to the sample.

73. A method of treating a neoplastic disease in a subject comprising:
administering to the subject a therapeutically effective amount of a senostimulator that increases a mean number of ATRX foci per cell at least 3-fold in a sample derived from the subject after the subject was administered a quiescence-inducing compound relative to a mean number of ATRX foci per cell in a reference sample collected from the subject before the administering of the senostimulator and after the administering of the quiescence-inducing compound.

74. A method of treating a neoplastic disease in a subject who has received a quiescence-inducing compound comprising:
administering to the subject a therapeutically effective amount of a senostimulator that
increases a number of ATRX foci per cell in a sample derived from the subject after the subject was administered the quiescence-inducing compound such that at least 30% of the cells have a number of ATRX foci that is at least 3 times higher than a mean number of ATRX foci per cell in a reference sample collected from the subject before the administering of the senostimulator and after the administering of the quiescence-inducing compound.

75. The method of claim 73 or 74, wherein the neoplastic disease is a cancer.

76. The method of any one of claims 73-75, wherein the administering of the senostimulator results in an increase in expression of one or more senescence-associated secretory phenotype (SASP) factors relative to the reference sample.

77. The method of claim 76, wherein the SASP factor is a growth arrest SASP factor.

78. The method of claim 77, wherein the growth arrest SASP factor is selected from the group consisting of TGF-β, IL-6, and IGFβ3.

79. The method of claim 76, wherein the SASP factor is an inflammatory SASP factor.

80. The method of claim 79, wherein the inflammatory SASP factor is selected from the group consisting of IL-1α, IL-1β, IL-8, and IFN-γ.

81. The method of claim 76, wherein the SASP factor is a stemness SASP factor.

82. The method of claim 81, wherein the stemness SASP factor is GM-CSE.

83. The method of claim 76, wherein the SASP factor is a remodeling of extracellular space SASP factor.

84. The method of claim 83, wherein the remodeling of extracellular space SASP factor is selected from the group consisting of MMP-1, MMP-3, MMP-10, MMP-12, MMP-13, and MMP-14.

85. The method of any one of claims 73-84, wherein the quiescence-inducing compound is a CDK4/6 inhibitor.

86. The method of claim 85, wherein the CDK4/6 inhibitor is PD0332991.

87. The method of any one of claims 73-86, wherein the quiescence-inducing compound is doxorubicin.

88. The method of any one of claims 73-87, wherein the quiescence-inducing compound causes a cancer cell to transition to quiescence.

89. The method of any one of claims 73-88, wherein the ATRX foci per cell are detected by immunofluorescence.

90. The method of claim 89, wherein the ATRX foci per cell are detected by an ATRX-specific antibody.

91. The method of claim 90, wherein the ATRX-specific antibody recognizes ATRX protein at the C-terminal domain.

92. The method of any one of claims 75-91, wherein the mean number of ATRX foci per cell is detected at least 1 day, at least 2 days, at least 3 days, at least 4 days, at least 5 days, at least 6 days, or at least 7 days after administering the quiescence-inducing compound to the subject.

93. A method of treating a subject comprising:
administering to the subject a senostimulator treatment, wherein the subject has previously received a quiescence-inducing compound treatment and a first senostimulator treatment resulting in at least a 3-fold increase in the mean number of ATRX foci per cell in a sample derived from the subject relative to a mean number of ATRX foci per cell in a reference sample collected from the subject prior to the first senostimulator treatment but after administration of the quiescence-inducing compound to the subject.

94. The method of claim 93, wherein the subject has one or more symptoms of a neoplastic disease.

95. The method of claim 93, wherein the neoplastic disease is a cancer.

96. The method of any one of claims 93-95, wherein the senostimulator causes an increase in expression of one or more senescence-associated secretory phenotype (SASP) factors relative to a reference sample.

97. The method of claim 96, wherein the SASP factor is a growth arrest SASP factor.

98. The method of claim 97, wherein the growth arrest SASP factor is selected from the group consisting of TGF-β, IL-6, and IGFβ3.

99. The method of claim 96, wherein the SASP factor is an inflammatory SASP factor.

100. The method of claim 99, wherein the inflammatory SASP factor is selected from the group consisting of IL-1α, IL-1β, IL-8, and IFN-γ.

101. The method of claim 96, wherein the SASP factor is a stemness SASP factor.

102. The method of claim 101, wherein the stemness SASP factor is GM-CSE.

103. The method of claim 96, wherein the SASP factor is a remodeling of extracellular space SASP factor.

104. The method of claim 103, wherein the remodeling of extracellular space SASP factor is selected from the group consisting of MMP-1, MMP-3, MMP-10, MMP-12, MMP-13, and MMP-14.

105. The method of any one of claims 93-104, wherein the senostimulator causes an increase in senescence-associated beta-galactosidase relative to the reference sample.

106. The method of any one of claims 93-105, wherein the quiescence-inducing compound is CDK4/6 inhibitor.

107. The method of claim 106, wherein the CDK4/6 inhibitor is PD0332991.

108. The method of any one of claims 93-104, wherein the quiescence-inducing compound is doxorubicin.

109. The method of any one of claims 93-108, wherein the quiescence-inducing compound causes a cancer cell to transition to quiescence.

110. The method of any one of claims 93-109, wherein the ATRX foci per cell are detected by immunofluorescence.

111. The method of claim 110, wherein the ATRX foci per cell are detected by an ATRX-specific antibody.

112. The method of claim 111, wherein the ATRX-specific antibody recognizes ATRX protein at the C-terminal domain.

113. The method of any one of claims 93-112, wherein the mean number of ATRX foci per cell is detected at least 1 day, at least 2 days, at least 3 days, at least 4 days, at least 5 days, at least 6 days, or at least 7 days after administering the quiescence-inducing compound to the subject.

114. The method of any one of claims 1-7, wherein the senosuppressor comprises a mixture of Ginsen, Yellow Qi, Manjing, Gegen, Yellow Po, Rising Ma, and Gan Weed.

115. The method of any one of claims 1-7, wherein the senosuppressor comprises a mixture of Ginsen, Stone CP, Jiuyuan, Fuling, and Fushen.

116. The method of any one of claims 1-7, wherein the senosuppressor comprises a mixture of Ginsen, Stone CP, Jiuyuan, Shudi, Shanzu, and Zhaoren.

117. The method of any one of claims 1-7, wherein the senosuppressor comprises a mixture of Ginsen, Jiuyuan, Fuling, Wuweizi, Zhaoren, Shengdi, Maidong, Tiandong, Dangqui, Pozi, Xuansen, Jigeng.
